(19) United States
(12) Patent Application Publication
(10) Pub. No.: US 2023/0341389 A1
(43) Pub. Date: Oct. 26, 2023

(54) COMPOSITIONS AND METHODS FOR DIAGNOSING PRIMARY BILIARY CIRRHOSIS

(71) Applicant: INOVA DIAGNOSTICS, INC., San Diego, CA (US)

(72) Inventors: GARY L. NORMAN, San Diego, CA (US); MICHAEL MAHLER, San Diego, CA (US)

(73) Assignee: INOVA DIAGNOSTICS, INC., San Diego, CA (US)

(21) Appl. No.: 18/007,700

(22) PCT Filed: Jan. 7, 2022
(86) PCT No.: PCT/US2022/011529

§ 371 Date: Dec. 1, 2022

Related U.S. Application Data
(60) Provisional application No. 63/135,469, filed on Jan. 8, 2021.

(51) Int. Cl. G01N 33/564; C07K 14/47

(52) U.S. Cl. CPC G01N 33/564; C07K 14/4713; G01N 2800/085; G01N 2333/4706

(57) ABSTRACT

The present disclosure relates to the use of anti-KLHL12 antibodies as a clinical biomarker for diagnosing primary biliary cirrhosis (PBC), now known as primary biliary cholangitis. The disclosure further provides compositions and methods for detecting anti-KLHL12 antibodies in a biological sample.

### CROSS-REFERENCE TO RELATED APPLICATIONS

This application claims the benefit of U.S. Application No. 63/135,469, filed Jan. 8, 2021, the disclosure of which is incorporated by reference herein in its entirety.

### REFERENCE TO SEQUENCE LISTING SUBMITTED ELECTRONICALLY

This application contains a sequence listing, which is submitted electronically via EFS-Web as an ASCII formatted sequence listing with a file name of “13510-035-228_SEQ_LISTING.txt” and a creation date of Jan. 2, 2022 and having a size of 6,613 bytes. The sequence listing submitted via EFS-Web is part of the specification and is herein incorporated by reference in its entirety.

### FIELD

The present disclosure relates to the field of autoimmune diseases and more particularly to compositions and methods for diagnosing primary biliary cirrhosis.

### BACKGROUND

Primary biliary cirrhosis (PBC), now known as primary biliary cholangitis, is a chronic, inflammatory, autoimmune disease of the liver. PBC is characterized by progressive damage to the bile ducts in the liver. Recent studies have identified the presence of circulating autoantibodies, which can be used as biomarkers for PBC. See Norman et al., Liver Int., 35(2):642-651 (2015); Hirschfield et al., Annu Rev Pathol., 8:303-330 (2013); Bogdanos et al., Dig Dis., 30 Suppl 1:20-31 (2012). However, a subgroup of PBC patients remain serologically negative for known autoantibodies and undiagnosed until more advanced disease has formed. Therefore, there is a need for new and/or more specific biomarkers to detect additional autoantibody species or increase the accuracy or sensitivity of diagnosis. The present disclosure addresses this need and provides related benefits as well.

### SUMMARY

In some embodiments, the present disclosure provides an isolated polypeptide comprising a Kelch-like 12 (KLHL12) fragment. The KLHL12 fragment comprises an amino acid sequence selected from the group consisting of SEQ ID NOS:1-25, wherein the amino acid sequence begins at the first recited amino acid residue.

In some embodiments, the KLHL12 fragment comprises 15-622 amino acid residues. In some embodiments, the KLHL12 fragment comprises 15, 20, 25, 30, 35, 40, 45 or 50 amino acid residues. In some embodiments, the KLHL12 fragment is 15 amino acid residues.

In some embodiments, the KLHL12 fragment is obtained by a method comprising isolation from a natural source, chemical synthesis or recombinant expression.

In some embodiments, the KLHL12 fragment comprises a solid support. In some embodiments, the solid support is selected from the group consisting of a bead, sphere, particle, membrane, chip, slide, plate, well and test tube. In some embodiments, the bead, sphere or particle comprises micrometer or nanometer dimensions. In some embodiments, the membrane is selected from the group consisting of nitrocellulose, nylon, polyvinylidene fluoride (PVDF) and polyvinylidene difluoride.

In some embodiments, the KLHL12 fragment specifically binds to an anti-KLHL12 antibody. In some embodiments, the anti-KLHL12 antibody comprises an autoantibody. In some embodiments, the binding exhibits a standard score for a signal-to-noise ratio of 25 or less (ZS).

In some embodiments, the present disclosure provides a kit. The kit comprises: (a) one or more isolated polypeptides comprising a KLHL12 fragment having an amino acid sequence selected from the group consisting of SEQ ID NOS:1-25, wherein the amino acid sequence begins at the first recited amino acid residue; and (b) a detection probe specific to an anti-KLHL12 antibody.

In some embodiments, the KLHL12 fragment in the kit comprises 15-622 amino acid residues. In some embodiments, the KLHL12 fragment in the kit comprises 15, 20, 25, 30, 35, 40, 45 or 50 amino acid residues. In some embodiments, the KLHL12 fragment in the kit is 15 amino acid residues.

In some embodiments, the KLHL12 fragment in the kit specifically binds to an anti-KLHL12 antibody. In some embodiments, the anti-KLHL12 antibody comprises an autoantibody. In some embodiments, the binding between the KLHL12 fragment and an anti-KLHL12 antibody exhibits a standard score for a signal-to-noise ratio of 25 or less (ZS).

In some embodiments, the KLHL12 fragment in the kit is obtained by a method comprising isolation from a natural source, chemical synthesis or recombinant expression.

In some embodiments, the detection probe comprises an antibody, an antibody specific binding polypeptide or a functional fragment of an antibody or an antibody specific binding polypeptide. In some embodiments, the antibody or function fragment thereof comprises anti-IgG. In some embodiments, the antibody specific binding polypeptide or functional fragment thereof comprises protein A or protein G.

In some embodiments, the detection probe further comprises a reporter tag. In some embodiment, the reporter tag is a label. In some embodiments, the label is selected from the group consisting of a fluorophore, enzyme, chemiluminescent moiety, radioactive moiety, organic dye and small molecule. In some embodiments, the label is a fluorescent label. In some embodiments, the fluorescent label is phycoerytherin (PE), horseradish peroxidase, alkaline phosphatase.

In some embodiments, the reporter tag comprises a ligand or particle. In some embodiments, the ligand is biotin. In some embodiments, the particle comprises a nanoparticle.

In some embodiments, the kit further comprises a solid support, control or ancillary reagent.

In some embodiments, the solid support is selected from the group consisting of a bead, sphere, particle, membrane, chip, slide, plate, well and test tube. In some embodiments, the bead, sphere or particle comprises micrometer or nanometer dimensions. In some embodiments, the membrane is selected from the group consisting of nitrocellulose, nylon, polyvinylidene fluoride (PVDF) and polyvinylidene difluoride.

In some embodiments, the one or more isolated polypeptide in the kit is conjugated to the solid support.

In some embodiments, the control comprises an antibody or functional fragment thereof specific to an isolated polypeptide having an amino acid sequence selected from the group consisting of SEQ ID NOS:1-25. In some embodiments, the antibody or functional fragment thereof is selected from a monoclonal or polyclonal antibody.

In some embodiments, the ancillary reagent is selected from the group consisting of incubation buffer, wash buffer, detection buffer and detection instrument.

In some embodiments, the present disclosure provides a method of diagnosing PBC. The method comprises: (a) contacting a biological sample from a subject suspected of having PBC with one or more isolated polypeptides comprising a KLHL12 fragment having amino acid sequence selected from the group consisting of SEQ ID NOS:1-25, and (b) detecting the presence of an anti-KLHL12 antibody in the biological sample, wherein the presence of the bound anti-KLHL12 antibody is indicative of PBC.

In some embodiments, the KLHL12 fragment in the method comprises 15-622 amino acid residues. In some embodiments, the KLHL12 fragment in the method comprises 15, 20, 25, 30, 35, 40, 45 or 50 amino acid residues. In some embodiments, the KLHL12 fragment in the method is 15 amino acid residues. In some embodiments, the amino acid sequence of the KLHL12 fragment in the method begins at the first recited amino acid residue.

In some embodiments, the KLHL12 fragment in the method specifically binds to an anti-KLHL12 antibody. In some embodiments, the anti-KLHL12 antibody comprises an autoantibody. In some embodiments, the binding between the KLHL12 fragment and an anti-KLHL12 antibody exhibits a standard score for a signal-to-noise ratio of 25 or less (ZS).

In some embodiments, the KLHL12 fragment in the method is obtained by a method comprising isolation from a natural source, chemical synthesis or recombinant expression.

In some embodiments, the biological sample comprises whole blood, plasma, serum, sputum or bile. In some embodiments, the biological sample comprises serum, plasma or sputum.

In some embodiments, the detection comprises an immunoassay. In some embodiments, the immunoassay is selected from the group consisting of a fluorescent immunosorbent assay (FIA), a chemiluminescent immunoassay (CIA), a radioimmunoassay (MA), multiplex immunoassay, a protein/peptide array immunoassay, a solid phase radioimmunoassay (SPRIA), an indirect immunofluorescence assay (IIF), an enzyme linked immunosorbent assay (ELISA) and a particle based multianalyte test (PMAT) or a Dot Blot assay.

In some embodiments, the detection comprises: (a) contacting the anti-KLHL12 antibody with a detection probe specific to the anti-KLHL12 antibody, and (b) detecting specific binding of the detection probe. In some embodiments, the detection probe comprises an antibody, an antibody specific binding polypeptide or functional fragment of an antibody or an antibody specific binding polypeptide. In some embodiments, the antibody or function fragment thereof comprises anti-IgG. In some embodiments, the antibody specific binding polypeptide or functional fragment thereof comprises protein A or protein G.

In some embodiments, the detection probe comprises a reporter tag. In some embodiment, the reporter tag is a label. In some embodiment, the label is selected from the group consisting of a fluorophore, enzyme, chemiluminescent moiety, radioactive moiety, organic dye and small molecule. In some embodiments, the label is a fluorescent label. In some embodiments, the fluorescent label is phycoerytherin (PE), horseradish peroxidase or alkaline phosphatase.

In some embodiments, the reporter tag comprises a ligand or particle. In some embodiments, the ligand is biotin. In some embodiments, the particle comprises a nanoparticle.

In some embodiments, the detection is performed on a solid support. In some embodiments, the solid support is selected from the group consisting of a bead, sphere, particle, membrane, chip, slide, plate, well and test tube. In some embodiments, the bead, sphere or particle comprises micrometer or nanometer dimensions. In some embodiments, the membrane is selected from the group consisting of nitrocellulose, nylon, polyvinylidene fluoride (PVDF) and polyvinylidene difluoride.

In some embodiments, the present disclosure provides a method of diagnosing PBC in a subject seronegative for known PBC autoantibodies. The method comprises: (a) contacting a biological sample from a subject seronegative for known PBC autoantibodies with one or more isolated polypeptides comprising a KLHL12 fragment having an amino acid sequence selected from the group consisting of SEQ ID NOS:1-25, and (b) detecting the presence of an anti-KLHL12 antibody in the biological sample bound to the one or more isolated polypeptides, wherein the presence of the bound anti-KLHL12 antibody is indicative of PBC.

In some embodiments, the subject is asymptomatic.

In some embodiments, the known PBC autoantibodies are selected from the group consisting of anti-mitochondrial antibodies (AMA), anti-nuclear antibodies (ANA), anti-multiple nuclear dots (MND) autoantibodies, anti-nuclear body (NB) autoantibodies, anti-hexokinase 1 (HK1) antibodies and anti-Kelch-like12 (KLHL12) antibodies. In some embodiments, the known PBC autoantibodies are selected from the group consisting of M2 mitochondrial autoantibody, gp230 autoantibody, nucleoporin p62 autoantibody, lamin B receptor autoantibody, promyelocytic leukemia protein (PML) autoantibody, anti-sp100 (Speckled) antibody, anti-gp210, anti-centromere, anti-97/VCP, anti-eosinophil peroxidase (anti-EPO), E2 subunits of the pyruvate dehydrogenase complex (PDC-E2) autoantibody, the branched/chain 2-oxo-acid dehydrogenase complex (BCOADC-E2) autoantibody, the 2-oxo-glutarate dehydrogenase complex (OGDC-E2) autoantibody and NDP52 autoantibody.

In some embodiments, the KLHL12 fragment in the method comprises 15-622 amino acid residues. In some embodiments, the KLHL12 fragment in the method comprises 15, 20, 25, 30, 35, 40, 45 or 50 amino acid residues. In some embodiments, the KLHL12 fragment in the method is 15 amino acid residues. In some embodiments, the amino acid sequence of the KLHL12 fragment in the method begins at the first recited amino acid residue.

In some embodiments, the KLHL12 fragment in the method specifically binds to an anti-KLHL12 antibody. In some embodiments, the anti-KLHL12 antibody comprises an autoantibody. In some embodiments, the binding between the KLHL12 fragment and an anti-KLHL12 antibody exhibits a standard score for a signal-to-noise ratio of 25 or less (ZS).

In some embodiments, the one or more isolated polypeptide in the method is obtained by a method comprising isolation from a natural source, chemical synthesis or recombinant expression.

In some embodiments, the biological sample comprises whole blood, plasma, serum, sputum or bile. In some embodiments, the biological sample comprises serum, plasma or sputum.

In some embodiments, the detection comprises an immunoassay. In some embodiments, the immunoassay is selected from the group consisting of a fluorescent immunosorbent assay (FIA), a chemiluminescent immunoassay (CIA), a radioimmunoassay (MA), multiplex immunoassay, a protein/peptide array immunoassay, a solid phase radioimmunoassay (SPRIA), an indirect immunofluorescence assay (IIF), an enzyme linked immunosorbent assay (ELISA) and a particle based multianalyte test (PMAT) or a Dot Blot assay.

In some embodiments, the detection comprises: (a) contacting the anti-KLHL12 antibody with a detection probe specific to the anti-KLHL12 antibody, and (b) detecting specific binding of the detection probe.

In some embodiments, the detection probe comprises an antibody, an antibody specific binding polypeptide or functional fragment of an antibody or an antibody specific binding polypeptide. In some embodiments, the antibody or function fragment thereof comprises anti-IgG. In some embodiments, the antibody specific binding polypeptide or functional fragment thereof comprises protein A or protein G.

In some embodiments, the detection probe comprises a reporter tag. In some embodiment, the reporter tag is a label. In some embodiment, the label is selected from the group consisting of a fluorophore, enzyme, chemiluminescent moiety, radioactive moiety, organic dye and small molecule. In some embodiments, the label is a fluorescent label. In some embodiments, the fluorescent label is phycoerytherin (PE), horseradish peroxidase or alkaline phosphatase.

In some embodiments, the reporter tag comprises a ligand or particle. In some embodiments, the ligand is biotin. In some embodiments, the particle comprises a nanoparticle.

In some embodiments, the detection is performed on a solid support. In some embodiments, the solid support is selected from the group consisting of a bead, sphere, particle, membrane, chip, slide, plate, well and test tube. In some embodiments, the bead, sphere or particle comprises micrometer or nanometer dimensions. In some embodiments, the membrane is selected from the group consisting of nitrocellulose, nylon, polyvinylidene fluoride (PVDF) and polyvinylidene difluoride.

### BRIEF DESCRIPTION OF THE DRAWINGS

FIG. 1 shows the sorting of various Kelch-like 12 (KLHL12) fragments based on either signal intensity analysis or signal-to-noise ratio analysis of the microarray scan result for epitope mapping of KLHL12 protein. Peptides with high signal intensity or high signal-to-noise ratio are further selected, synthesized and evaluated via several solid-phase assays, including ELISA and bead-based technologies.

FIG. 2 shows a receiver operating characteristic (ROC) analysis of KLHL12 fragment P2623-1 (square), KLHL12 fragment P2738-1 (triangle) and commercially produced Cyclic Citrullinated Peptide (CCP) (diamond), illustrating their respective capacity to specifically bind anti-KLHL12 antibodies. The amino acid sequences of P2623-1 and P2738-1 are both SEQ ID NO:1. The difference is that P2623-1 is labeled with biotin at the C-terminus of SEQ ID NO:1 and bound to Costar high-binding microwell plate, while P2938-1 is labeled at the N-terminus of SEQ ID NO:1 and bound to Inova streptavidin coated plate. CCP is an antigen for antibodies present in many patients with rheumatoid arthritis. The CCP coated plate used herein is obtained from QUANTA Lite® CCP3 IgG ELISA kits (Inova Diagnostics, San Diego, CA). Area Under the Curve (AUC) for each marker is shown in the figure legend.

FIG. 3 shows a ROC analysis of KLHL12 fragment P2623-1 (dark gray), KLHL12 fragment P2738-1 (black) and Abnova KLHL12 (light gray), illustrating their respective capacity to specifically bind anti-KLHL12 antibodies. The KLHL12 fragments P2623-1 and P2738-1 used in FIG. 3 are the same as those used in FIG. 2. Abnova KLHL12 is a full-length recombinant KLHL12 protein with GST-tag at the N-terminus. It is commercially obtained from Abnova Corporation, Taipei, Taiwan. AUC is indicated in parentheses in the figure legend.

FIG. 4 shows the overlap of anti-MIT3, anti-HK-1 and anti-KLHL12 antibody testing using a Venn diagram. The addition of anti-HK-1 and anti-KLHL12 antibody testing to anti-MIT3 antibody testing increased the sensitivity of detecting Primary Biliary Cirrhosis (PBC) from 85.0% for anti-MIT3 alone to 90.8% for the combination of anti-MIT3, anti-HK-1 and anti-KLHL12 for the sample cohort. KL-p refers to the KLHL12 fragment used in the assay.

FIG. 5 shows the frequency and overlap of autoimmune liver disease-associated antibodies in a cohort of French patients with PBC. KL-p refers to the KLHL12 fragment used in the assay.

FIG. 6 shows the prevalence of anti-HK-1, anti-KLHL12, anti-gp210, anti-sp100, anti-SLA, and anti-LC1 antibodies in 8 MIT3-negative patients with PBC. KL-p refers to the KLHL12 fragment used in the assay.

### DETAILED DESCRIPTION

The present disclosure is based, in part, on the discovery of Kelch-like 12 (KLHL12) epitopes that are specific to autoantibodies directed against KLHL12 protein and can be used to detect anti-KLHL12 antibodies as an indicator of primary biliary cirrhosis (PBC), now known as primary biliary cholangitis. Thus, the present disclosure benefits PBC subjects by specific detection of anti-KLHL12 antibodies that are indicative of the presence of PBC including, for example, in subjects that are seronegative for known biomarkers of PBC. Such benefits further enable at risk or early-stage PBC subjects to prevent or reduce disease progression and related symptoms.

It must be noted that, as used in this specification and the appended claims, the singular forms “a,” “an” and “the” also include plural referents unless the content clearly dictates otherwise.

It must also be noted that, as used in this specification and the appended claims, where a range of numeric values is provided, it is understood that the ranges are inclusive of the numbers defining the range. It is also understood that each intervening integer within the recited range as well as fractions thereof, including for example, every tenth of a unit of a selected intervening integer or a lower limit of the recited range is intended to be included within the disclosure, unless the context clearly dictates otherwise.

As used herein, the terms “comprises,” “comprising,” “includes,” “including,” “has,” “having,” “contains,” “containing,” and any variations thereof, are intended to cover a non-exclusive inclusion, such that a process, method, product-by-process or composition of matter that includes, has or contains an element or list of elements does not include only those elements but can include other elements not expressly listed or inherent to such process, method, product-by-process or composition of matter.

The present disclosure provides an isolated polypeptide having a KLHL12 fragment. The KLHL12 fragment can have an amino acid sequence selected from the group consisting of SEQ ID NOS:1-25, wherein the amino acid sequence begins at the first recited amino acid residue. The isolated KLHL12 fragment can be included in the kits provided herein or used in the methods provided herein.

As used herein, the term “Kelch-like 12” or “KLHL12,” also known as Kelch like Family Member 12, DKIR, HDKIR, CUL3-Interacting Protein 1, C3IP1 and Kelch-like Protein C3IP1, refers to a polypeptide which acts as a substrate adaptor of the Cullin-3 ubiquitin ligase complex to promote ubiquitination. See Rondou et al., J Biol Chem., 283:11083-11096, (2008). KLHL12 is expressed in various tissues including the liver, pancreas, gall bladder, colon, adrenal tissues and small intestine, to name a few. An exemplary human KLHL12 nucleotide sequence can be found in GenBank under GenBank GI number 1676325197 and encodes an exemplary human KLHL12 having the amino acid sequence found in GenBank under GenBank GI number 733606608. The GenBank GI numbers of this KLHL12 and other KLHL12 isoforms can be found below in Table 1, which contains two sequence identifiers, the GI number and the GenBank accession number. The GI number and GenBank accession number run in parallel as unique identifiers to access the referenced sequence in publicly available databases.

TABLE 1

| Molecule Type | GI Number | GenBank Accession Number |
|---|---|---|
| Homo sapiens kelch like family member 12 (KLHL12), | 1676325197 | NM_001303051.2 |
| transcript variant 1, mRNA | | |
| kelch-like protein 12 isoform 1 [Homo sapiens] | 733606608 | NP_001289980.1 |
| Homo sapiens kelch like family member 12 (KLHL12), | 1519241658 | NM_021633.4 |
| transcript variant 2, mRNA | | |
| kelch-like protein 12 isoform 2 [Homo sapiens] | 11056006 | NP_067646.1 |
| Homo sapiens kelch like family member 12 (KLHL12), | 733606702 | NM_001303109.1 |
| transcript variant 3, mRNA | | |
| kelch-like protein 12 isoform 3 [Homo sapiens] | 733606703 | NP_001290038.1 |
| PREDICTED: Homo sapiens kelch like family member | 1370454090 | XM_011509835.2 |
| 12 (KLHL12), transcript variant X1, mRNA | | |
| kelch-like protein 12 isoform X1 [Homo sapiens] | 767910065 | XP_011508137.1 |
| PREDICTED: Homo sapiens kelch like family member | 1370454091 | XM_011509836.2 |
| 12 (KLHL12), transcript variant X2, mRNA | | |
| kelch-like protein 12 isoform X2 [Homo sapiens] | 767910067 | XP_011508138.1 |
| PREDICTED: Homo sapiens kelch like family member | 1370454092 | XM_011509837.2 |
| 12 (KLHL12), transcript variant X3, mRNA | | |
| kelch-like protein 12 isoform X3 [Homo sapiens] | 767910069 | XP_011508139.1 |
| PREDICTED: Homo sapiens kelch like family member | 1370454093 | XM_017001995.2 |
| 12 (KLHL12), transcript variant X4, mRNA | | |
| kelch-like protein 12 isoform X4 [Homo sapiens] | 1034560779 | XP_016857484.1 |
| Homo sapiens mRNA for KLHL12 variant, clone: | 62897704 | AK223072.1 |
| KAT02609 | | |
| KLHL12 variant, partial [Homo sapiens] | 62897705 | BAD96792.1 |
| KLHL12 (Drosophila), isoform CRA_e, partial [Homo | 119611854 | EAW91448.1 |
| sapiens] | | |
| Chain A, KELCH-LIKE PROTEIN 12 | 170292438 | 2VPJ_A |

Approximately 476 coding single nucleotide polymorphisms (SNPs) and at least 257 known orthologs have been identified for KLHL12 (see, for example, NCBI Gene ID: 59349). All of such KLHL12 polypeptides and variants thereof are included within the meaning of the term “KLHL12” as it is used herein.

It should be understood that a variant refers to a nucleic acid or amino acid sequence that is similar but differs from the wild-type sequence by at least one nucleotide or amino acid. A wild-type nucleic acid or amino acid sequence refers to those nucleic acid and amino acid sequences prevalent among a population and serve as a reference for its respective variants.

The present disclosure provides isolated polypeptides having KLHL12 fragments that are antigenic epitopes recognized by anti-KLHL12 antibodies, including anti-KLHL12 human autoantibodies. Accordingly, the present disclosure provides an isolated KLHL12 fragment having an amino acid sequence selected from CYDPIIDSWEVVTSM (SEQ ID NO:1), IECYDPIIDSWEVVT (SEQ ID NO:2), YDPIIDSWEVVTSMG (SEQ ID NO:3), SIECYDPIIDSWEVV (SEQ ID NO:4), ECYDPIIDSWEVVTS (SEQ ID NO:5), VVASGVIYCLGGYDG (SEQ ID NO:6), GHWTNVTPMATKRSG (SEQ ID NO:7), AGVALLNDHIYVVGG (SEQ ID NO:8), ASGVIYCLGGYDGLN (SEQ ID NO:9), GAGVALLNDHIYVVG (SEQ ID NO:10), MGGIMAPKDIMTNTH (SEQ ID NO:11), GLVVASGVIYCLGGY (SEQ ID NO:12), SGVIYCLGGYDGLNI (SEQ ID NO:13), LQYVRMPLLTPRYIT (SEQ ID NO:14), MTTPRCYVGATVLRG (SEQ ID NO:15), GLAGATTLGDMIYVS (SEQ ID NO:16), RIYVIGGYDGRSRLS (SEQ ID NO:17), ECLDYTADEDGVWYS (SEQ ID NO:18), GVWYSVAPMNVRRGL (SEQ ID NO:19), DSWTTVTSMTTPRCY (SEQ ID NO:20), YNIRTDSWTTVTSMT (SEQ ID NO:21), GGFDGSRRHTSMERY (SEQ ID NO:22), RSGAGVALLNDHIYV (SEQ ID NO:23), LNDHIYVVGGFDGTA (SEQ ID NO:24), or IECYDPIIDSWEVVTSMG (SEQ ID NO:25). In some embodiments, the isolated KLHL12 fragments begin at the first recited amino acid sequence. In some embodiments, the isolated KLHL12 fragments end at the last recited amino acid residue. In other embodiments, the isolated KLHL12 fragments both begin and end at the recited amino acid residues.

The present disclosure further provides an isolated KLHL12 fragment consisting of an amino acid sequence selected from CYDPIIDSWEVVTSM (SEQ ID NO:1), IECYDPIIDSWEVVT (SEQ ID NO:2), YDPIIDSWEVVTSMG (SEQ ID NO:3), SIECYDPIIDSWEVV (SEQ ID NO:4), ECYDPIIDSWEVVTS (SEQ ID NO:5), VVASGVIYCLGGYDG (SEQ ID NO:6), GHWTNVTPMATKRSG (SEQ ID NO:7), AGVALLNDHIYVVGG (SEQ ID NO:8), ASGVIYCLGGYDGLN (SEQ ID NO:9), GAGVALLNDHIYVVG (SEQ ID NO:10), MGGIMAPKDIMTNTH (SEQ ID NO:11), GLVVASGVIYCLGGY (SEQ ID NO:12), SGVIYCLGGYDGLNI (SEQ ID NO:13), LQYVRMPLLTPRYIT (SEQ ID NO:14), MTTPRCYVGATVLRG (SEQ ID NO:15), GLAGATTLGDMIYVS (SEQ ID NO:16), RIYVIGGYDGRSRLS (SEQ ID NO:17), ECLDYTADEDGVWYS (SEQ ID NO:18), GVWYSVAPMNVRRGL (SEQ ID NO:19), DSWTTVTSMTTPRCY (SEQ ID NO:20), YNIRTDSWTTVTSMT (SEQ ID NO:21), GGFDGSRRHTSMERY (SEQ ID NO:22), RSGAGVALLNDHIYV (SEQ ID NO:23), LNDHIYVVGGFDGTA (SEQ ID NO:24), or IECYDPIIDSWEVVTSMG (SEQ ID NO:25).

SEQ ID NO:1 of the above exemplary isolated KLHL12 fragments corresponds to amino acids 577-591 of human KLHL12 isoform 1, the sequence of which can be found in GenBank under GenBank GI number 733606608. SEQ ID NO:2 of the above exemplary isolated KLHL12 fragments corresponds to amino acids 575-589 of human KLHL12 isoform 1. SEQ ID NO:3 of the above exemplary isolated KLHL12 fragments corresponds to amino acids 578-592 of human KLHL12 isoform 1. SEQ ID NO:4 of the above exemplary isolated KLHL12 fragments corresponds to amino acids 574-588 of human KLHL12 isoform 1. SEQ ID NO:5 of the above exemplary isolated KLHL12 fragments corresponds to amino acids 576-590 of human KLHL12 isoform 1. SEQ ID NO:6 of the above exemplary isolated KLHL12 fragments corresponds to amino acids 460-474 of human KLHL12 isoform 1. SEQ ID NO:7 of the above exemplary isolated KLHL12 fragments corresponds to amino acids 489-503 of human KLHL12 isoform 1. SEQ ID NO:8 of the above exemplary isolated KLHL12 fragments corresponds to amino acids 504-518 of human KLHL12 isoform 1. SEQ ID NO:9 of the above exemplary isolated KLHL12 fragments corresponds to amino acids 462-476 of human KLHL12 isoform 1. SEQ ID NO:10 of the above exemplary isolated KLHL12 fragments corresponds to amino acids 503-517 of human KLHL12 isoform 1. SEQ ID NO:11 of the above exemplary isolated KLHL12 fragments corresponds to amino acids 39-53 of human KLHL12 isoform 1. SEQ ID NO:12 of the above exemplary isolated KLHL12 fragments corresponds to amino acids 458-472 of human KLHL12 isoform 1. SEQ ID NO:13 of the above exemplary isolated KLHL12 fragments corresponds to amino acids 463-477 of human KLHL12 isoform 1. SEQ ID NO:14 of the above exemplary isolated KLHL12 fragments corresponds to amino acids 257-271 of human KLHL12 isoform 1. SEQ ID NO:15 of the above exemplary isolated KLHL12 fragments corresponds to amino acids 544-558 of human KLHL12 isoform 1. SEQ ID NO:16 of the above exemplary isolated KLHL12 fragments corresponds to amino acids 408-422 of human KLHL12 isoform 1. SEQ ID NO:17 of the above exemplary isolated KLHL12 fragments corresponds to amino acids 368-382 of human KLHL12 isoform 1. SEQ ID NO:18 of the above exemplary isolated KLHL12 fragments corresponds to amino acids 385-399 of human KLHL12 isoform 1. SEQ ID NO:19 of the above exemplary isolated KLHL12 fragments corresponds to amino acids 395-409 of human KLHL12 isoform 1. SEQ ID NO:20 of the above exemplary isolated KLHL12 fragments corresponds to amino acids 536-550 of human KLHL12 isoform 1. SEQ ID NO:21 of the above exemplary isolated KLHL12 fragments corresponds to amino acids 531-545 of human KLHL12 isoform 1. SEQ ID NO:22 of the above exemplary isolated KLHL12 fragments corresponds to amino acids 423-437 of human KLHL12 isoform 1. SEQ ID NO:23 of the above exemplary isolated KLHL12 fragments corresponds to amino acids 501-515 of human KLHL12 isoform 1. SEQ ID NO:24 of the above exemplary isolated KLHL12 fragments corresponds to amino acids 509-523 of human KLHL12 isoform 1. SEQ ID NO:25 of the above exemplary isolated KLHL12 fragments corresponds to amino acids 575-592 of human KLHL12 isoform 1.

The nucleotide sequence of SEQ ID NO:1 of the above exemplary isolated KLHL12 fragments corresponds to nucleotides 1779-1823 of Homo sapiens KLHL12 transcript variant 1, the sequence of which can be found in GenBank under GenBank GI number 1676325197. The nucleotide sequence of SEQ ID NO:2 of the above exemplary isolated KLHL12 fragments corresponds to nucleotides 1773-1817 of Homo sapiens KLHL12 transcript variant 1. The nucleotide sequence of SEQ ID NO:3 of the above exemplary isolated KLHL12 fragments corresponds to nucleotides 1782-1826 of Homo sapiens KLHL12 transcript variant 1. The nucleotide sequence of SEQ ID NO:4 of the above exemplary isolated KLHL12 fragments corresponds to nucleotides 1770-1814 of Homo sapiens KLHL12 transcript variant 1. The nucleotide sequence of SEQ ID NO:5 of the above exemplary isolated KLHL12 fragments corresponds to nucleotides 1776-1820 of Homo sapiens KLHL12 transcript variant 1. The nucleotide sequence of SEQ ID NO:6 of the above exemplary isolated KLHL12 fragments corresponds to nucleotides 1428-1472 of Homo sapiens KLHL12 transcript variant 1. The nucleotide sequence of SEQ ID NO:7 of the above exemplary isolated KLHL12 fragments corresponds to nucleotides 1515-1559 of Homo sapiens KLHL12 transcript variant 1. The nucleotide sequence of SEQ ID NO:8 of the above exemplary isolated KLHL12 fragments corresponds to nucleotides 1560-1604 of Homo sapiens KLHL12 transcript variant 1. The nucleotide sequence of SEQ ID NO:9 of the above exemplary isolated KLHL12 fragments corresponds to nucleotides 1434-1478 of Homo sapiens KLHL12 transcript variant 1. The nucleotide sequence of SEQ ID NO:10 of the above exemplary isolated KLHL12 fragments corresponds to nucleotides 1557-1601 of Homo sapiens KLHL12 transcript variant 1. The nucleotide sequence of SEQ ID NO:11 of the above exemplary isolated KLHL12 fragments corresponds to nucleotides 165-209 of Homo sapiens KLHL12 transcript variant 1. The nucleotide sequence of SEQ ID NO:12 of the above exemplary isolated KLHL12 fragments corresponds to nucleotides 1422-1466 of Homo sapiens KLHL12 transcript variant 1. The nucleotide sequence of SEQ ID NO:13 of the above exemplary isolated KLHL12 fragments corresponds to nucleotides 1437-1481 of Homo sapiens KLHL12 transcript variant 1. The nucleotide sequence of SEQ ID NO:14 of the above exemplary isolated KLHL12 fragments corresponds to nucleotides 819-863 of Homo sapiens KLHL12 transcript variant 1. The nucleotide sequence of SEQ ID NO:15 of the above exemplary isolated KLHL12 fragments corresponds to nucleotides 1680-1724 of Homo sapiens KLHL12 transcript variant 1. The nucleotide sequence of SEQ ID NO:16 of the above exemplary isolated KLHL12 fragments corresponds to nucleotides 1272-1316 of Homo sapiens KLHL12 transcript variant 1. The nucleotide sequence of SEQ ID NO:17 of the above exemplary isolated KLHL12 fragments corresponds to nucleotides 1152-1196 of Homo sapiens KLHL12 transcript variant 1. The nucleotide sequence of SEQ ID NO:18 of the above exemplary isolated KLHL12 fragments corresponds to nucleotides 1203-1247 of Homo sapiens KLHL12 transcript variant 1. The nucleotide sequence of SEQ ID NO:19 of the above exemplary isolated KLHL12 fragments corresponds to nucleotides 1233-1277 of Homo sapiens KLHL12 transcript variant 1. The nucleotide sequence of SEQ ID NO:20 of the above exemplary isolated KLHL12 fragments corresponds to nucleotides 1656-1700 of Homo sapiens KLHL12 transcript variant 1. The nucleotide sequence of SEQ ID NO:21 of the above exemplary isolated KLHL12 fragments corresponds to nucleotides 1641-1685 of Homo sapiens KLHL12 transcript variant 1. The nucleotide sequence of SEQ ID NO:22 of the above exemplary isolated KLHL12 fragments corresponds to nucleotides 1317-1361 of Homo sapiens KLHL12 transcript variant 1. The nucleotide sequence of SEQ ID NO:23 of the above exemplary isolated KLHL12 fragments corresponds to nucleotides 1551-1595 of Homo sapiens KLHL12 transcript variant 1. The nucleotide sequence of SEQ ID NO:24 of the above exemplary isolated KLHL12 fragments corresponds to nucleotides 1575-1619 of Homo sapiens KLHL12 transcript variant 1. The nucleotide sequence of SEQ ID NO:25 of the above exemplary isolated KLHL12 fragments corresponds to nucleotides 1773-1826 of Homo sapiens KLHL12 transcript variant 1.

It should be noted that “polypeptide” can include a short oligopeptide having between 2 and 30 amino acids (e.g., 2, 3, 4, 5, 6, 7, 8, 9, 10, 12, 14, 16, 18, 20, 25 or 30 amino acids) as well as longer amino acid chains, e.g., more than 30 amino acids, more than 50 amino acids, more than 100 amino acids, more than 150 amino acids, more than 200 amino acids, more than 300 amino acids, more than 400 amino acids, more than 500 amino acids or more than 600 amino acids. Such short oligopeptides are referred to herein as KLHL12 fragments.

The isolated KLHL12 fragment disclosed herein is less than the full-length KLHL12. In some embodiments, the isolated KLHL12 fragment shares a partial amino acid sequence of full-length KLHL12 which can include 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more or 25 or more consecutive amino acids of the full-length KLHL12.

In some embodiments, the isolated KLHL12 fragment can be of any mammalian origin. In other embodiments, the isolated KLHL12 fragment can be of human origin.

As used herein, the term “isolated” when used in reference to a polypeptide is intended to mean that the polypeptide can be partially or substantially isolated from a complex mixture of components. The complex mixture can include, for example, a source where the polypeptide is chemically synthesized, recombinantly expressed or naturally expressed. Partial isolation includes isolation from one or more components whereas substantial isolation includes isolation from, for example, many, most or substantially all components from the polypeptide's source.

Partial isolation, as disclosed herein, can be achieved by the methods and compositions provided herein. In some embodiments, a partially isolated KLHL12 fragment can be performed with a capture probe. In some embodiments, the capture probe is a polypeptide or functional fragment thereof specific to the KLHL12 fragment. In some embodiments, the capture probe is an anti-KLHL12 antibody. Substantial isolation, as exemplified herein, can be achieved by methods known in the art. In some embodiments, an isolated KLHL12 fragment is purified substantially by a process of extraction, precipitation and solubilization.

The isolated KLHL12 fragment can be chemically synthesized, recombinantly synthesized or isolated from a natural source such as a bodily fluid or tissue. Exemplary methods for expressing and isolating chemically synthesized polypeptides, recombinant polypeptides or polypeptides from a natural source are well known in the art and can be found described in Scopes R. K., Protein Purification—Principles and Practice, Springer Advanced Texts in Chemistry, 3rd Edition (1994); Simpson R. J. et al., Basic Methods in Protein Purification and Analysis: A Laboratory Manual, Cold Spring Harbor Laboratory Press, 1st Edition (2008); Green M. R. and Sambrook J., Molecular Cloning: A Laboratory Manual, Cold Spring Harbor Laboratory Press, 4th Edition (2012); Jensen K. J. et al., Peptide Synthesis and Applications (Methods in Molecular Biology), Humana Press, 2nd Edition (2013).

Recombinant polypeptides can be expressed in and purified from bacterial cells (e.g., E. coli), yeast cells (e.g., S. cerevisiae), insect cells (e.g., S f 9), in mammalian cells (e.g., CHO) and others. Recombinant polypeptides can be expressed and purified as fusion proteins including tags for protein detection or affinity purification tags (e.g., His-tag, GST-tag, Myc-tag), including cleavable tags (e.g., tags including a TEV-cleavage site). One skilled in the art will recognize that methods for the purification of polypeptides from cells, tissues or bodily fluids are well known in the art.

Polypeptides purified or isolated from a natural source refers to the isolation and purification of a polypeptide from a source where it is naturally expressed. In some embodiments, the isolated KLHL12 fragment can be from a cell, tissue or bodily fluid of an organism. In some embodiments, the cells, tissues or bodily fluids can include, for example, whole blood, plasma, serum, sputum or bile from an organism of the present disclosure. The isolated KLHL12 fragment can similarly be from any biological sample described and provided herein.

In some embodiments, the isolated KLHL12 fragment is chemically synthesized using, for example, methods described in Jensen, K. J. (supra).

In some embodiments, the isolated KLHL12 fragment can be a fragment of a native KLHL12. In some embodiments, the isolated KLHL12 fragment can be a fragment of a denatured or unfolded KLHL12. In some embodiments, the isolated KLHL12 fragment can include unnatural amino acids. In some embodiments, the unnatural amino acids can be methylated at the α-amino-group to produce polypeptides with methylated backbones. In some embodiments, the unnatural amino acids can be R-amino acids. In some embodiments, the unnatural amino acids can include dyes (e.g., fluorescent dyes) or affinity tags. In some embodiments, the isolated KLHL12 fragment can include chemical modifications. Chemical modifications can include, e.g., chemical modifications with biotin, fluorescent dyes. A skilled artisan will recognize that methods for introducing unnatural amino acids into polypeptides and for chemically modifying polypeptides are well known in the art.

In some embodiments, an isolated, chemically synthesized or recombinant KLHL12 fragment can be a plurality of the KLHL12 fragments disclosed herein. It should be noted that the term “plurality” refers to a population of two or more members, such as polypeptide members or other referenced molecules. In some embodiments, the two or more members of a plurality of members can be the same members. For example, a plurality of polypeptides can include two or more polypeptide members having the same amino acid sequence. By way of exemplification, a plurality of members having the same amino acid sequence can include two or more members of any one of the isolated KLHL12 fragments of SEQ ID NOS:1-25. In some embodiments, the two or more members of a plurality of members can be different members. For example, a plurality of polypeptides can include two or more polypeptide members having different amino acid sequences. By way of exemplification, a plurality of members having different amino acid sequences can include at least two or more of the isolated KLHL12 fragments of SEQ ID NOS:1-25. A plurality can includes 2, 3, 4, 5, 6, 7, 8, 9, 10, 20, 30, 40, 50, 60, 70, 80, 90 or a 100 or more different members. A plurality can also include 200, 300, 400, 500, 1000, 5000 or 10000 or more different members. A plurality includes, for example, all integer numbers in between the above exemplary plurality numbers. In some embodiments, the isolated KLHL12 fragment can be a plurality of KLHL12 fragments from the organisms of the present disclosure.

As used herein, the terms “solid support,” “solid surface” and other grammatical equivalents refer to any material that is appropriate for or can be modified to be appropriate for the attachment of isolated KLHL12 fragments of this disclosure. Possible materials include, for example, glass and modified or functionalized glass, plastics (including acrylics, polystyrene, methylstyrene, polyurethanes, Teflon″, etc.), paramagnetic materials, thoria sol, carbon graphite, titanium oxide, latex or cross-linked dextrans such as Sepharose, cellulose polysaccharides, nylon or nitrocellulose, ceramics, resins, silica or silica-based materials including silicon and modified silicon, carbon metals, inorganic glasses, optical fiber bundles and a variety of other polymers. As an example, a solid support of the present disclosure can include a multiwell plate such as a 96-, 384- or 1536-well plate. In other examples, the solid supports can be located within a flow cell or flow cell apparatus (e.g., a flow cell on a Biacore™ chip or a protein chip). Exemplary solid supports provided herein can also be used in the methods and kits of the disclosure.

In some embodiments, the solid support can be a bead, sphere, particle, membrane, chip, slide, well and test tube. Beads include spheres or particles. By “spheres” or “particles” or grammatical equivalents herein is meant small, discrete, non-planar particles in the micrometer or nanometer dimensions. The spheres can include, for example, microspheres. The particles can include, for example, nanoparticles. In some embodiments the bead can be spherical, in other embodiments the bead is irregular. Alternatively or additionally, the beads can be porous. The bead sizes range from nanometers to millimeters with beads from about 0.2 to about 200 microns being preferred in some embodiments. In other embodiments, bead size can range from about 0.5 to about 5 microns. In some embodiments, beads smaller than 0.2 microns and larger than 200 microns can be used. In some embodiments, the solid support can include an array of wells or depressions in a surface. This can be fabricated as is known in the art using a variety of techniques, including, photolithography, stamping techniques, molding techniques and microetching techniques. As will be appreciated by those skilled in the art, the technique used will depend on the composition and shape of the array substrate.

In some embodiments, the solid support can include a patterned surface suitable for immobilization of purified proteins in an ordered pattern (e.g., a protein chip). A “patterned surface” refers to an arrangement of different regions in or on an exposed layer of a solid support. For example, one or more of the regions can be features where one or more purified proteins are present. The features can be separated by interstitial regions where purified proteins are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. Exemplary patterned surfaces that can be used in the methods and compositions set forth herein are described in U.S. Pat. App. Publ. No. 2008/0280785 A1, U.S. Pat. App. Publ. No. 2004/0253640 A1, U.S. Pat. App. Publ. No. 2003/0153013 A1 and International Publication No. WO 2009/039170 A2.

In some embodiments, a solid support can have attached to its surface the isolated KLHL12 fragment. Any isolated KLHL12 fragment exemplified by including an amino acid sequence selected from the group consisting of SEQ ID NOS:1-25 can be attached to a solid support. In some embodiments, any isolated KLHL12 fragment can be immobilized to a solid support via a linker molecule. In some embodiments, all that is required is that molecules, such as any isolated KLHL12 fragment, remain immobilized or attached to the support under the conditions in which it is intended to use the support, for example, in applications requiring antibody binding or detection. It should be noted that the term “immobilized” is used interchangeably with “attached” and both terms are intended to include covalent and non-covalent attachment, unless indicated otherwise, either explicitly or by context.

As used herein, the term “antibody” is used interchangeably with immunoglobulin (Ig) and refers to a polypeptide product of B-cells or recombinant equivalents thereof, that is able to bind to a specific molecular antigen and is composed of two heavy chains and two light chains. Each amino-terminal portion of each chain includes a variable region that confers binding specificity. See Borrebaeck (ed.), Antibody Engineering, Second Edition, Oxford University Press. (1995); Kuby, Immunology, Third Edition, W.H. Freeman and Company, New York (1997). The term includes autoantibodies and antibodies used as detection probes in the methods and kits disclosed herein. The antibody can exhibit specific binding affinity where it binds to a single molecular species or pan-specific where it binds selectively to more than one related molecular species. In the context of the present disclosure, the specific molecular antigen that can be bound by an antibody of the disclosure includes, for example, any of the KLHL12 fragments having SEQ ID NOS:1-25 or an anti-KLHL12 antibody including, for example, an anti-KLHL12 antibody specific to any one or more of the KLHL12 fragments having SEQ ID NOS:1-25. An antibody of the present disclosure can be derived from any mammalian organism, including mouse, rabbit, goat, chicken, donkey and the like. Furthermore, a primary or secondary antibody can be monoclonal, polyclonal, chimeric or humanized. The antibodies provided herein can also be used in the methods and kits of the disclosure.

As used herein, the term “anti-KLHL12 antibody” when used in reference to an autoantibody, is intended to mean an autoantibody specific or pan-specific to one or more KLHL12 fragments having SEQ ID NOS:1-25. An anti-KLHL12 antibody can include any antibody class (IgG, IgM, IgD, IgA and IgE) and subclass (IgG1, IgG2, IgG3, IgG4, IgA1 and IgA2) or a functional fragment thereof. In some embodiments, an anti-KLHL12 antibody includes a human autoantibody to human KLHL12.

It should be understood that the term “autoantibody” refers to an immunoglobulin directed against a constituent of tissue of the subject that produces the autoantibody. The term is intended to include an antibody produced by a subject's immune system that is directed against one or more of the subject's own polypeptides or antigens. Accordingly, autoantibodies can be produced by a subject's immune system when the immune system fails to distinguish in whole or in part, between self and non-self tissue constituents. As provided herein, exemplary autoantibodies include an anti-KLHL12 antibody as described herein.

As used herein, the term “signal-to-noise ratio” refers to the ratio between one protein's specific binding affinity to its target and the protein's non-specific binding affinity to a non-target. One skilled in the art will recognize that methods for measuring the binding affinity between proteins are well known in the art. In some embodiments, the binding between an isolated KLHL12 fragment and an anti-KLHL12 antibody, including an anti-KLHL12 autoantibody, exhibits a standard score for a signal-to-noise ratio of 25 or less (ZS).

The present disclosure provides a kit for diagnosing PBC. The kit can include one or more isolated KLHL12 fragments of the present disclosure. Exemplary isolated KLHL12 fragments include SEQ ID NOS:1-25 disclosed herein and found in Table 2 below. In some embodiments, the isolated KLHL12 fragments begin at the first recited amino acid sequence. In some embodiments, the isolated KLHL12 fragments end at the last recite amino acid residue. In other embodiments, the isolated KLHL12 fragments both begin and end at the recited amino acid residue. The isolated KLHL12 fragment can be chemically synthesized, recombinantly synthesized or isolated from a natural source as disclosed herein. The isolated KLHL12 fragment can specifically bind to an anti-KLHL12 antibody, including an anti-KLHL12 autoantibody, where the binding exhibits a standard score for a signal-to-noise ratio of 25 or less (ZS).

The kit can include a detection probe specific to an anti-KLHL12 antibody. As used herein, the term “detection probe” refers to a binding agent capable of specific binding to a target. Such binding agents include, for example, antibodies and antibody specific binding polypeptides. The detection probes provided herein can also be used in the methods of the disclosure.

Antibodies include full length antibodies as well as functional fragments such as those exemplified below. As used herein, the term “functional fragment,” when used in reference to an antibody, is intended to refer to a portion of the antibody including heavy or light chain polypeptides that retain some or all of the binding activity as the antibody from which the fragment was derived. Such functional fragments can include, for example, an Fd, Fv, Fab, F(ab′), F(ab) 2, F(ab′) 2, single chain Fv (scFv), diabody, triabody, tetrabody and minibody, which can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Plückthun and Skerra, Meth. Enzymol., 178:497-515 (1989) and in Day, E. D., Advanced Immunochemistry, Second Ed., Wiley-Liss, Inc., New York, NY (1990). The antibody functional fragments provided herein can be used in the methods and compositions of the disclosure.

Antibody specific binding polypeptides are provided herein and include any polypeptide that can specifically recognize antibodies as well as functional fragments thereof. Exemplary antibody specific binding polypeptides include IgG binding proteins, receptors, chimeric receptors and binding polypeptides identified from screening of random or combinatorial libraries. Exemplary antibody specific binding polypeptides of the present disclosure include KLHL12 or an antigenic fragment thereof or IgG binding proteins. Exemplary IgG binding proteins include protein A and protein G. Antibody specific binding polypeptides of the present disclosure can be obtained or synthesized by methods described herein or known in the art, including for example, chemically synthesized, purified from a natural source or recombinantly made. Thus, antibody specific binding polypeptides described herein can be mammalian, including mouse, rabbit, goat, chicken, donkey and the like. All of such antibody specific binding polypeptides provided herein can be used in the methods and compositions of the disclosure.

When referring to specific binding to a target, a detection probe of the disclosure can bind the target directly or it can be made specific to the target by indirect means. For example, a detection probe that binds directly to an anti-KLHL12 antibody includes KLHL12 or any of the isolated KLHL12 fragments disclosed herein. A direct binder also includes, for example, an antibody or other antibody specific binding polypeptide that specifically recognizes a KLHL12:anti-KLHL12 antibody complex as well as an antibody or other antibody specific binding polypeptide that specifically binds to an anti-KLHL12 antibody. A detection probe of the disclosure that can be made specific to the target by indirect means can include, for example, anti-Ig or other antibody specific binding polypeptide that binds Ig. Such antibodies and antibody specific binding polypeptides can be made specific to anti-KLHL12 antibody by, for example, capturing the anti-KLHL12 antibody with an isolated KLHL12 fragment described herein and washing away non-anti-KLHL12 Ig prior to adding anti-Ig or other antibody specific binding polypeptide that binds Ig. Numerous other configurations for isolating or separating such a binding complex in order to achieve specific binding to a target are well known in the art and all of which can be used as an indirect means to make a detection probe specific to a target. Thus, a “detection probe specific to an anti-KLHL12 antibody” includes, for example, KLHL12, a KLHL12:anti-KLHL12 antibody complex binding agent, an anti-KLHL12 antibody binding agent and an Ig binding agent.

Accordingly, in some embodiments, the antibody specific to an anti-KLHL12 antibody is goat anti-human IgG or a functional fragment thereof. In some embodiments, the binding polypeptide specific to an anti-KLHL12 antibody is protein A or protein G or functional fragment thereof.

The detection probe can include a reporter tag. As used herein, the term “reporter tag” refers to a molecule capable of producing a signal indicative of the detection of a biomarker. An exemplary biomarker in the present disclosure includes anti-KLHL12 antibody. Reporter tags can be attached or conjugated, for example, to the detection probe through non-covalent or covalent cross-linkage. Non-covalent and covalent immobilization of reporter tags to detection probes can be performed by any means known in the art, including methods as described in, for example, Dennler et al., “Antibody conjugates: from heterogeneous populations to defined reagents,” Antibodies. 4:197-224 (2015). Reporter tags produce various signals, depending on the type of reporter tag. A person skilled in the art appreciates that there are various labels encompassed by reporter tags.

As used herein, the term “label” refers to a molecular entity that emits a signal and can be used as a readout or measurement for detection of an analyte. Various classes of labels exist. Examples of these classes include a flurophore, an enzyme, a chemiluminscent moiety, a radioactive moiety, an organic dye, a small molecule, a polypeptide or functional fragment thereof. Examples of fluorophores include fluorescent dyes like phycoerytherin (PE), fluorescein isothiocyanate (FITC), tetramethylrhodamine (TRITC), BODIPY and AlexaFluor® dyes. Fluorescent dyes can also include fluorescence resonance energy transfer (FRET)-dyes or time-resolved (TR)-FRET dyes. Fluorophore labels also include fluorescent proteins such as green fluorescent protein (GFP) and cyan fluorescent protein (CFP). Examples of enzyme labels include alkaline phosphatase (AP) or horseradish peroxidase (HRP). When any of the substrates 3,3′5,5′-Tetramethylbenzidine (TMB), 3,3′-Diaminobenzidene (DAB) or 2,2′-azino-bis(3-ethylbenzothiazoline-6-sulphonic acid) (ABTS) are applied to HRP, a colored (chromogenic) or light (chemiluminescent) signal is produced. Radioactive labels include carbon-14 or Tritium, for example. Small molecule labels include biotin, resins such as agarose beads and fluorescently labeled magnetic beads or nanoparticles such as colloidal gold. Polypeptide or functional fragment labels include Avidin, Streptavidin or NeutrAvidin which have an affinity for biotin. Polypeptide or functional fragment labels also include hemagglutinin (HA), glutathione-S-transferase (GST) or c-myc. The reporter tags and labels provided herein can also be used in the methods of the disclosure.

A label of the present disclosure can be conjugated to any of the detection probes identified herein. Conjugation can include non-covalent or covalent cross-linkage as described above. In some configurations, a label conjugated to a detection probe requires an additional substrate or binding agent described above. As an example, an HRP label conjugated to a detection probe requires a substrate, disclosed above, to detect a detection probe. Numerous other configurations for a label are known in the art. The present disclosure includes all label configurations exemplified herein and/or known in the art. In some embodiments, a label configuration can include PE conjugated to an isolated KLHL12 fragment having an amino acid sequence selected from the group consisting of SEQ ID NOS:1-25, a KLHL12:anti-KLHL12 antibody complex binding agent, an anti-KLHL12 antibody binding agent or an Ig binding agent.

The kit can include a solid support. Exemplary solid support includes a bead, sphere, particle, membrane, chip, slide, plate, well and test tube as disclosed herein. In some embodiments, the bead, sphere or particle comprises micrometer or nanometer dimensions. In some embodiments, the membrane is selected from the group consisting of nitrocellulose, nylon, polyvinylidene fluoride (PVDF) and polyvinylidene difluoride. Any isolated KLHL12 fragment exemplified by including an amino acid sequence selected from the group consisting of SEQ ID NOS:1-25 can be attached to a solid support as disclosed herein.

The kit can include control. As used herein, “control” refers to a positive or negative standard for comparison against a tested sample where the existence or amount of anti-KLHL12 antibody is unknown. A kit can include a positive control. In some embodiments, a positive control can be a sample containing a detectable amount of anti-KLHL12 antibody or functional fragment thereof or levels above the threshold. In some embodiments, a positive control can be obtained from a diseased subject who has levels of anti-KLHL12 antibody above threshold. Additionally or alternatively, a positive control can contain antibody or functional fragment thereof specific to an isolated KLHL12 fragment having an amino acid sequence selected from the group consisting of SEQ ID NOS:1-25 synthesized in vitro using any of the methods described herein. The antibody or functional fragment thereof can be selected from a monoclonal or polyclonal antibody. In other embodiments, the kit can include a negative control. A negative control can be a sample containing no detectable amount of anti-KLHL12 antibody or functional fragment thereof or levels below the threshold. In some embodiments, a negative control can be obtained from a healthy control individual or can be synthesized in vitro. For example, a negative control can include water or buffer.

The kit can include one or more ancillary reagents. As used herein, “ancillary reagents” refer to a substance, mixture, material or component that is useful to carry out an intended purpose of a composition or method of the disclosure. Exemplary ancillary reagents include a conjugation reagent, a buffer, instructions, instruments and the like. A person skilled in the art recognizes that there are various types of incubation, washing, detection and blocking buffers. The reagents provided herein can also be used in the methods of the disclosure.

In some embodiments, a reagent of the kit of the present disclosure can include any conjugation reagent known in the art, including covalent and non-covalent conjugation reagents. Covalent conjugation reagents can include any chemical or biological reagent that can be used to covalently immobilize a polypeptide of this disclosure on a surface. Covalent conjugation reagents can include a carboxyl-to-amine reactive group such as carbodiimides such as EDC or DCC, an amine reactive group such as N-hydroxysuccinimide (NETS) ester or imidoesters, a sulfhydryl-reactive crosslinker such as maleimides, haloacetyls or pyridyl disulfides, a carbonyl-reactive crosslinker groups such as, hydrazides or alkoxyamines, a photoreactive crosslinker such as aryl azides or dizirines or a chemoselective ligation group such as a Staudinger reaction pair. Non-covalent immobilization reagents can include any chemical or biological reagent that can be used to immobilize a polypeptide of this disclosure non-covalently on a surface, such as affinity tags such as biotin or capture reagents such as streptavidin or anti-tag antibodies, such as anti-His6 or anti-Myc antibodies.

The kits of this disclosure can include combinations of conjugation reagents. Such combinations include, e.g., EDC and NHS, which can be used, e.g., to immobilize a protein of this disclosure on a surface, such as a carboxylated dextrane matrix (e.g., on a BIAcore™ CM5 chip or a dextrane-based bead). Combinations of conjugation reagents can be stored as premixed reagent combinations or with one or more conjugation reagents of the combination being stored separately from other conjugation reagents.

In other embodiments, a reagent of the kit can include a reagent such as a coating buffer. A coating buffer can include sodium carbonate-sodium hydroxide or phosphate. In some embodiments, the coating buffer can be 0.1M NaHCO₃(e.g., about pH 9.6).

In some embodiments, a reagent of a kit can include a wash buffer. A wash buffer can include tris (hydroxymethyl)aminomethane (Tris)-based buffers like Tris-buffered saline (TB S) or phosphate buffers like phosphate-buffered saline (PBS). Wash buffers can be composed of detergents, such as ionic or non-ionic detergents. In some embodiments, the wash buffer can be a PBS buffer at about pH 7.4 including Tween® 20 at about 0.05%. In other embodiments, the wash buffer can be the BIO-FLASH™ Special Wash Solution (Inova Diagnostics, Inc., San Diego, CA).

In some embodiments, a reagent of the kit can include a dilution buffer. Any dilution buffer known in the art can be included in the kit of the present disclosure. Typical dilution buffers include a carrier protein such as bovine serum albumin (BSA) and a detergent such as Tween®20. In some embodiments, the dilution buffer can be PBS at about pH 7.4 including BSA at about 1% BSA and Tween® 20 at about 0.05%.

In some embodiments, a reagent can include a detection or assay buffer. Any detection or assay buffer known in the art can be included in the kit of the present disclosure. The detection or assay buffer can be a colorimetric detection or assay buffer, a fluorescent detection or assay buffer or a chemiluminescent detection or assay buffer. Colorimetric detection or assay buffers include PNPP (p-nitrophenyl phosphate), ABTS (2,2′-azino-bis(3-ethylbenzothiazoline-6-sulphonic acid)) or OPD (o-phenylenediamine). Fluorescent detection or assay buffers include QuantaBlu™ or QuantaRed™ (Thermo Scientific, Waltham, MA). Chemiluminescent detection or assay buffers can include luminol or luciferin. Detection or assay buffers can also include a trigger such as H₂O₂ and a tracer such as isoluminol-conjugate. In some embodiments, the detection reagent can include one or more BIO-FLASH™ Trigger solutions (Inova Diagnostics, Inc., San Diego, CA). In some embodiments, a reagent of the kit of the present disclosure can include solutions useful for calibration or testing.

In some embodiments, a reagent of the kit can include a stop solution. Any stop solution known in the art can be included in a kit of this disclosure. The stop solutions of this disclosure terminate or delay the further development of the detection reagent and corresponding assay signals. Stop solutions can include, e.g., low-pH buffers (e.g., glycine-buffer, pH 2.0), chaotrophic agents (e.g., guanidinium chloride, sodium-dodecylsulfate (SDS)) or reducing agents (e.g., dithiothreitol, β-mecaptoethanol) or the like.

In some embodiments, a reagent of the kit of this disclosure can include cleaning reagents. Cleaning reagents can include any cleaning reagent known in the art. In some embodiments, the cleaning reagents can be the cleaning reagents recommended by the manufacturers of the automated assay systems. In some embodiments, the cleaning reagents can include the BIO-FLASH™ System Rinse or the BIO-FLASH™ System Cleaning solutions (Inova Diagnostics, Inc., San Diego, CA).

In some embodiments, a kit of the disclosure can include an instrument to an automated assay system. Automated assay systems can include systems by any manufacturer. In some embodiments, the automated assay systems can include, e.g., the BIO-FLASH™, the QUANTA-Lyser® 4000, QUANTA-Lyser® 3000, QUANTA-Lyser® 160, QUANTA-Lyser® 240, QUANTA-Lyser® 2 and the DS2™ (Inova Diagnostics, Inc., San Diego, CA). In some embodiments, the automated assay systems can include, e.g., the BEST 2000™, the ELx50 WASHER, the ELx800 READER, and the Autoblot S20™ (Biokit, Barcelona, Spain). In other embodiments, an instrument of the kit can be a detection instrument. A detection instrument can include any detection instrument in the art. Detection instruments are capable of detecting or measuring a label of the reporter tags of the present disclosure. Thus, detection instruments are capable of detecting or measuring fluorescence, luminescence, chemiluminescence or absorbance, reflectance, transmittance, birefringence or refractive index. In some embodiments, detection instruments can include confocal and non-confocal microscopy, a microplate reader, a flow cytometer and the like.

In some embodiments, the kit provided in this disclosure can include a component suitable for collecting a biological sample as described below. A component can include collection tubes, columns, syringes, needles and the like. In some embodiments, the kit can include instructions for using the components of the kit. Instructions can be in any form, inside or outside of the kit. The instructions provide details regarding protocol and analytical techniques.

Components of a kit of the disclosure can be in varying physical states. For example, some or all of the components can be lyophilized or in aqueous solution or frozen. Such components include an isolated KLHL12 fragment, a detection probe and ancillary reagents.

A kit of this disclosure can be tailored to specific assay technologies. In some embodiments, a kit can be tailored to assay technologies exemplified herein. For example, in some embodiments, the kits can be a FIA kit, a CIA kit, a RIA kit, a multiplex immunoassay kit, a protein/peptide array immunoassay kit, a SPRIA kit, an IIF kit, an ELISA, a PMAT kit or a Dot Blot kit. In some embodiments, the ELSA kits can include a washing buffer, a sample diluent, a secondary antibody-enzyme conjugate, a detection reagent and a stop solution. In some embodiments, the Dot Blot kits can include a washing buffer, a sample diluent, a secondary antibody-enzyme conjugate, a detection reagent and a stop solution. In some embodiments, the CIA kit can include a washing buffer, a sample diluent, a tracer (e.g., isoluminol-conjugate) and a trigger (e.g., H₂O₂). In some embodiments, the multiplex kit can include a washing buffer, a sample diluent and a secondary antibody-enzyme conjugate. In some embodiments, the kits can be tailored to the Luminex platform and include, as an example, xMAP® beads.

A kit can be used to diagnose PBC by providing a means for detecting anti-KLHL12 antibody bound to an isolated KLHL12 fragment. A kit can detect anti-KLHL12 antibody by any of the methods disclosed herein (see below). The KLHL12:anti-KLHL12 antibody complex or antigenic fragment thereof can have a stoichiometry of one to one or more than one to one anti-KLHL12 antibody. In some embodiments, the complexes can have one anti-KLHL12 antibody per isolated KLHL12 fragment. In some embodiments, the complexes can have two anti-KLHL12 antibodies per isolated KLHL12 fragment. In some embodiments, the complexes can have more than two anti-KLHL12 antibodies per isolated KLHL12 fragment. Methods for measuring binding stoichiometries of two antigens are well known in the art and include, e.g., isothermal titration calorimetry (ITC) and ultracentrifugation.

In some embodiments, the KLHL12:anti-KLHL12 antibody complex or antigenic fragment thereof, can be a plurality of complexes with identical stoichiometry. For example, all complexes in the plurality of complexes have one anti-KLHL12 antibody per isolated KLHL12 fragment. In some embodiments, the KLHL12:anti-KLHL12 antibody complex or antigenic fragment thereof, can be a plurality of complexes with different stoichiometries. For example, some complexes in the plurality of complexes can have one anti-KLHL12 antibody per isolated KLHL12 fragment and some other complexes in the plurality of complexes can have more than one anti-KLHL12 antibody per isolated KLHL12 fragment.

In some embodiments, an isolated KLHL12 fragment can be bound by anti-KLHL12 antibody with higher affinity. In some embodiments, anti-KLHL12 antibody binding sites can be bound by anti-KLHL12 antibody with more than 2-fold, more than 3-fold, more than 4-fold, more than 5-fold, more than 8-fold, more than 10-fold, more than 15-fold, more than 20-fold, more than 25-fold, more than 50-fold, more than 100-fold, more than 300-fold, more than 1,000-fold, more than 3,000-fold, more than 10,000-fold, more than 30,000-fold or more than 100,000-fold greater binding affinity. Greater binding affinities are evidenced by lower dissociation constants (K_{DS}) for the KLHL12:anti-KLHL12 antibody complex or by higher association constants (K_{AS}) for the respective anti-KLHL12 antibody and KLHL12. In some embodiments, the dissociation constants for (K_{DS}) for the KLHL12:anti-KLHL12 antibody complex can be less than 1 mM, less than 300 nM, less than 100 nM, less than 30 nM, less than 10 nM, less than 3 nM, less than 1 nM, less than 300 pM, less than 100 pM, less than 30 pM, less than 10 pM, less than 3 pM or less than 1 pM. Methods for measuring binding affinities of antibodies to antigens are well known in the art and include ELISA, isothermal titration calorimetry (ITC) and surface plasmon resonance (SPR).

The present disclosure provides methods for diagnosing PBC, including in a subject seronegative for known PBC antibodies. Such methods include: (a) contacting a biological sample from a subject suspected of having PBC, including a subject seronegative for known PBC antibodies, with an isolated KLHL12 fragment described herein, and (b) detecting the presence of an anti-KLHL12 antibody in the biological sample, wherein the presence of the bound anti-KLHL12 antibody is indicative of PBC.

The presence of increased anti-KLHL12 antibody in a subject compared to a healthy control individual can be indicative of the presence of PBC or the risk of developing PBC. Accordingly, a measurable increase in an autoantibody to KLHL12 is used to diagnose PBC. Exemplary methods for detection and comparison of anti-KLHL12 antibody levels to a control are provided herein and described further below.

In some embodiments, the level of anti-KLHL12 antibody is detected. In other embodiments, KLHL12:anti-KLHL12 antibody complex can be formed using the compositions and methods described herein and an anti-KLHL12 antibody in the complex can be detected.

In some embodiments, detection of an increased level of anti-KLHL12 antibody compared to a healthy control individual is indicative of a subject having PBC. In some embodiments, following diagnosis of PBC using the compositions and methods provided herein, the presence of PBC can be further corroborated based on a variety of symptoms associated with the onset or presence of PBC. Clinical symptoms associated with PBC include, for example, fatigue, pruritus, sicca syndrome and upper abdominal discomfort. See Norman et al., Liver Int., 35(2):642-651 (2015); Onofrio et al., Gastroenterol Hepatol (N Y), 15(3):145-154 (2019).

In some embodiments, detection of an increased level of anti-KLHL12 antibody compared to a healthy control individual indicates that the subject is at risk of developing clinical symptoms of PBC. In some embodiments, a subject can be at risk of developing clinical symptoms of PBC within less than 3 months, less than 6 months, less than 9 months, less than 12 months, less than 18 months, less than 2 years, less than 3 years, less than 4 years, less than 5 years, less than 6 years, less than 7 years, less than 8 years, less than 9 years, less than 10 years, less than 12 years, less than 14 years or less than 16 years from the determination of the increased anti-KLHL12 antibody level.

In some embodiments, the presence of an increased level of anti-KLHL12 antibody compared to a healthy control individual indicates that the subject is more than 5%, more than 10%, more than 15%, more than 20%, more than 25%, more than 30%, more than 35%, more than 40%, more than 45%, more than 50%, more than 60%, more than 70% or more than 80% or more than 90% likely to develop clinical symptoms of PBC within 5 years following the determination of increased anti-KLHL12 antibody. In some embodiments, the presence of an increased level of anti-KLHL12 antibody can indicate that the subject is more than 2-fold, more than 3-fold, more than 4-fold, more than 5-fold, more than 6-fold, more than 7-fold, more than 8-fold, more than 9-fold or more than 10-fold likely to develop clinical symptoms of PBC within 5 years following determination of increased anti-KLHL12 antibody level compared to a healthy control individual.

As used herein, the terms “subject,” “organism,” “individual” or “patient” are used as synonyms and interchangeably and refer to a vertebrate mammal. Mammals include humans, primates such as monkeys, chimpanzees, orangutans and gorillas, cats, dogs, rabbits, farm animals such as cows, horses, goats, sheep and pigs and rodents such as mice, rats, hamsters and guinea pigs. As an example, the subjects of this disclosure can include healthy subjects, asymptomatic subjects and diseased subjects.

As used herein, a subject can be “suspected of having PBC” as determined by the presence of certain risk factors that are well known in the art. Such risk factors include, for example, a genetic predisposition, a personal disease history, a lifestyle factor, an environmental factor, a diagnostic indicator and the like.

In some embodiments, a subject can be suspected of having PBC based on the presence of certain risk factors that are well known in the art. A subject at risk for having or developing PBC can have a genetic predisposition for developing PBC or a family history of PBC or other autoimmune diseases. The subject can be exposed to certain lifestyle factors (e.g., smoking cigarettes) or environmental factors promoting the development of PBC. The subject can show clinical disease manifestations of PBC. The subject can be a patient who is receiving a clinical workup to diagnose PBC or to assess the risk of developing PBC.

In some embodiments, the subjects can have anti-KLHL12 antibodies in their bodily fluid or tissue. In some embodiments, subjects can have elevated anti-KLHL12 antibodies in their bodily fluid or tissue compared to normal healthy subjects. In some embodiments, the subjects will not have elevated anti-KLHL12 antibodies in their bodily fluid or tissue compared to normal healthy subjects.

In some embodiments, the subject can be treatment naïve. In some embodiments, the subject can be undergoing treatments for PBC (e.g., drug treatments). In some embodiments, the subject can be in remission. In some embodiments, the remission can be drug-induced. In some embodiments, the remission can be drug-free.

In some embodiments, the subject can be an animal model for PBC. In some embodiments, the animal model can be a mouse, rabbit or primate model of PBC. In some embodiments, the animal model can involve inducing anti-KLHL12 antibody responses by immunizing or vaccinating an animal with KLHL12.

As used herein, a subject can be “seronegative for known PBC autoantibodies” as determined by the failure to detect the presence of known PBC autoantibodies in a blood test.

In some embodiments, the known PBC antibodies can be anti-mitochondrial antibodies (AMA), anti-nuclear antibodies (ANA), anti-multiple nuclear dots (MND) autoantibodies, anti-nuclear body (NB) autoantibodies, anti-hexokinase 1 (HK1) antibodies and anti-KLHL12 antibodies. In some embodiments, the known PBC antibodies can be M2 mitochondrial autoantibody, gp230 autoantibody, nucleoporin p62 autoantibody, lamin B receptor autoantibody, promyelocytic leukemia protein (PML) autoantibody, anti-sp100 (Speckled) antibody, anti-gp210, anti-centromere, anti-97/VCP, anti-eosinophil peroxidase (anti-EPO), E2 subunits of the pyruvate dehydrogenase complex (PDC-E2) autoantibody, the branched/chain 2-oxo-acid dehydrogenase complex (BCOADC-E2) autoantibody, the 2-oxo-glutarate dehydrogenase complex (OGDC-E2) autoantibody and NDP52 autoantibody.

In some embodiments, the subject can be asymptomatic. Asymptomatic subjects include healthy subjects who have essentially no risk or only a low risk of developing PBC (e.g., there is a less than 10%, less than 5%, less than 3% or less than 1% probability that the asymptomatic patient will develop PBC over the following five year period). Asymptomatic subjects further include healthy subjects who have a high risk of developing PBC (e.g., there is a greater than 50%, greater than 70%, greater than 90% or greater than 95% probability that the asymptomatic patient will develop PBC over the following five year period). Asymptomatic subjects further include diseased subjects, who can display mild early diagnostic indicators of PBC, but who are otherwise disease or complaint free.

It should be noted that the terms “healthy control individual,” “healthy subjects,” and grammatical equivalents herein are used interchangeably and refer to subjects who do not have increased anti-KLHL12 antibody above baseline or a standard known or determined to represent non-PBC subjects.

In some embodiments, the healthy subjects can have never suffered from a certain disease. In some embodiments, the healthy subjects can be previously diseased. In some embodiments, the healthy subjects can be undergoing a routine medical checkup. In some embodiments, the healthy subjects can be members of a control group in, for example, a clinical trial. In some embodiments, the healthy subjects can be at risk of contracting a disease, as determined by the presence of certain risk factors that are well known in the art. Such risk factors include, without limitation, a genetic predisposition, a personal disease history, a familial disease history, a lifestyle factor, an environmental factor, a diagnostic indicator and the like.

The baseline or standard which determines or defines a subject as a non-PBC subject is the reference interval. In diagnostic or health-related fields, the reference interval is a range of values observed in the reference subjects, which can be healthy control individuals, designated by specific percentiles. The reference interval can be any range of values as determined by those having skill in the art. See CLSI, “How to define and determine reference intervals in the clinical laboratory: approved guideline,” C28:A2 (2000). In some cases, the reference interval can be stringent or less stringent depending on the specific analyte being measured or disease being studied. A person having skill in the art will understand the appropriate stringency to use when determining the reference interval. Thus, in some embodiments, the reference interval can be set at the 95th percentile. In order to increase specificity and decrease sensitivity, e.g. increase stringency, a higher cut-off can be used such as the 96th percentile or the 97th or the 98^{th} or the 99th.

In the present disclosure, anti-KLHL12 antibody can be considered increased in a subject if anti-KLHL12 antibody levels are at least above the 95th percentile relative to anti-KLHL12 antibody levels in healthy control subjects. In other embodiments, anti-KLHL12 antibody can be considered increased in a subject if anti-KLHL12 antibody levels are above the 96^{th}, 97^{th}, 98^{th} or 99^{th} percentile. A subject of the present disclosure with anti-KLHL12 antibody levels at or above any of the disclosed reference intervals is considered to have PBC.

In some embodiments, the presence of anti-KLHL12 antibody can be based on a comparison of signal against background in a healthy subject. In some embodiments, the presence of anti-KLHL12 antibody can be increased or decreased relative to an average or median anti-KLHL12 antibody level observed in a population of healthy subjects. In some embodiments, anti-KLHL12 antibody can be absent in healthy subjects. In some embodiments, anti-KLHL12 antibody level cannot be detected above the noise of the respective assay used to determine anti-KLHL12 antibody level. In some embodiments, anti-KLHL12 antibody can be considered present in a sample if an anti-KLHL12 antibody level can be detected above the noise of the respective assay used to determine an anti-KLHL12 antibody level. In some embodiments, anti-KLHL12 antibody can be considered increased in a sample if the signal in an anti-KLHL12 antibody detection assay is at least two standard deviations above noise such as the average or mean signal for control samples. In some embodiments, anti-KLHL12 antibody can be considered present in a sample if the level of anti-KLHL12 antibody exceeds a predetermined threshold level. An anti-KLHL12 antibody threshold level can be determined by a skilled artisan, such as a clinical physician, based on a variety of factors, such as the specific objectives of a clinical trial or the diagnostic and prognostic significance of a certain anti-KLHL12 antibody level or the results of another diagnostic test for PBC that does not involve the detection of anti-KLHL12 antibody levels.

Anti-KLHL12 antibodies can be detected in a variety of different biological samples obtained from a subject. As used herein, the term “biological sample” refers to any specimen from the body of an organism that can be used for analysis or diagnosis. As an example, a biological sample can include a liquid sample such as whole blood, plasma, serum, synovial fluid, amniotic fluid, sputum, pleural fluid, peritoneal fluid, central spinal fluid, urine, bile, saliva or tears. A biological sample can also include a solid tissue sample such as a liver biopsy, bone marrow, buccal or other solid or semi-solid aggregated of cells. In the context of the present disclosure, a biological sample obtained from a subject can be any sample that contains or is suspected to contain autoantibodies and encompasses any material from a subject in which an anti-KLHL12 antibody can be detected.

The biological sample of the present disclosure can be obtained from any subject that contains or is suspected to contain anti-KLHL12 antibodies. A biological sample of the present disclosure can also be obtained from any subject that does not or is not suspected to have anti-KLHL12 antibodies. Thus, in some embodiments, a biological sample can be obtained from a symptomatic subject, an asymptomatic subject and a subject that is negative for anti-KLHL12 antibodies. In further embodiments, the biological sample can be obtained from mammals such as human, primates such as monkeys, chimpanzees, orangutans and gorillas, cats, dogs, rabbits, farm animals such as cows, horses, goats, sheep and pigs and rodents such as mice, rats, hamsters and guinea pigs.

A biological sample of the present disclosure can be collected and processed immediately or it can be collected, frozen and processed at a later date. The biological sample could also include a plurality of samples from one of the subjects described herein. In some embodiments, the plurality of biological samples can be collected over periodically over the course of more than 12 hours, more than 1 day, more than 2 days, more than 3 days, more than 4 days, more than 5 days, more than 6 days, more than 7 days, more than 10 days, more than 14 days, more than 3 weeks, more than 1 month, more than 2 months, more than 3 months, more than 4 months, more than 5 months, more than 6 months, more than 9 months, more than 12 months, more than 18 months, more than 24 months, more than 30 months, more than 3 years months, more than 4 years or more than 5 years.

A biological sample of the present disclosure can be whole blood, serum, plasma or sputum. In some embodiments, a biological sample of the present disclosure can be a tissue biopsy such as a liver biopsy. The liver biopsy can be less than 10 mm, less than 11 mm, less than 12 mm, less than 13 mm, less than 14 mm, less than 15 mm, less than 16 mm, less than 17 mm, less than 18 mm, less than 19 mm, less than 20 mm, less than 21 mm, less than 22 mm, less than 21 mm, less than 23 mm, less than 24 mm or less than 25 mm in length. The liver biopsy can include, in addition to the lengths disclosed herein, at least 4 portal triads, at least 5 portal triads, at least 6 portal triads, at least 7 portal triads, at least 8 portal triads, at least 9 portal triads, at least 10 portal triads or at least 11 portal triads.

The methods provided herein can include one or more isolated KLHL12 fragments of the present disclosure. Exemplary isolated KLHL12 fragments include SEQ ID NOS:1-25 disclosed herein and found in Table 2 below. In some embodiments, the isolated KLHL12 fragments begin at the first recited amino acid sequence. In some embodiments, the isolated KLHL12 fragments end at the last recite amino acid residue. In other embodiments, the isolated KLHL12 fragments both begin and end at the recited amino acid residue. The isolated KLHL12 fragment can be chemically synthesized, recombinantly synthesized or isolated from a natural source as disclosed herein. The isolated KLHL12 fragment can specifically bind to an anti-KLHL12 antibody, including an anti-KLHL12 autoantibody, where the binding exhibits a standard score for a signal-to-noise ratio of 25 or less (ZS).

In some embodiments, the method described herein can be performed by contacting the biological sample with 1 or more, 2 or more, 3 or more, 4 or more, 5 or more, 6 or more, 7 or more, 8 or more, 9 or more, 10 or more, 11 or more, 12 or more, 13 or more, 14 or more, 15 or more or 16 or more of the isolated KLHL12 fragments disclosed herein. In some embodiments, the 1 or more isolated KLHL12 fragments are conjugated to any of the solid supports disclosed herein. In other embodiments, the 1 or more isolated KLHL12 fragments are not conjugated to any of the solid supports disclosed herein.

The detection of the presence of the bound anti-KLHL12 antibodies includes contacting anti-KLHL12 antibodies with a detection probe specific to anti-KLHL12 antibodies and detecting specific binding of the detection probe.

A detection probe of the method can include any of the detection probes described above. In brief, detection probes of the method can include antibodies and antibody specific binding polypeptides or functional fragment of antibodies or antibody specific binding polypeptides. Thus, a detection probe specific for anti-KLHL12 antibody includes, for example, an isolated KLHL12 fragment, a KLHL12:anti-KLHL12 antibody complex binding agent, an anti-KLHL12 antibody binding agent and an Ig binding agent.

In some embodiments, the antibody specific to an anti-KLHL12 antibody is goat anti-human IgG or a functional fragment thereof. In some embodiments, the binding polypeptide specific to an anti-KLHL12 antibody is protein A or protein G or functional fragment thereof.

As provided herein and exemplified with respect to the kits of this disclosure, a method of this disclosure can include a reporter tag. Reporter tags function to produce a signal for detection of a biomarker. In some embodiments, the reporter tag includes a ligand or particle. In some embodiments, the ligand is biotin. In some embodiments, the particle includes a nanoparticle. Reporter tags can be attached, for example, to any of the detection probes used herein through non-covalent or covalent cross-linkage. As exemplified with respect to the kits of this disclosure, a method of this disclosure can include any of the labels described or exemplified herein. For example, a label of the kit can include a fluorophore, an enzyme, a chemiluminescent moiety, a radioactive moiety, an organic dye, a small molecule, a polypeptide or functional fragment thereof. In some embodiments, a label of the kit includes a fluorescent label. In some embodiments, the fluorescent label is PE, horseradish peroxidase or alkaline phosphatase. In some embodiments, a label of the present disclosure is conjugated to a detection probe of the disclosure as exemplified above.

As provided herein, PBC can be determined in subject of the present disclosure by detecting the presence of the bound anti-KLHL12 antibodies. Methods for detecting, measuring and/or quantifying a signal produced by a label of the present disclosure are well known in the art and include detection of fluorescence, luminescence, chemiluminescence or absorbance, reflectance, transmittance, birefringence or refractive index. Optical methods include imaging methods such as confocal and non-confocal microscopy and non-imaging methods such as microplate readers. In some embodiments, methods of detecting anti-KLHL12 antibody in biological sample can include visualization, quantification or both of a fluorescent, colorimetric or absorbance signal in a biological sample.

In some embodiments of the present disclosure, anti-KLHL12 antibody presence can be detected by immunoassay. Methods and protocols for conducting immunoassays and biophysical protein-interaction assays are well known in the art. See, e.g., Wild D., The Immunoassay Handbook, Elsevier Science, 4^{th} Edition (2013); Fu H., Protein-Protein Interactions, Humana Press, 4^{th} Edition (2004). Exemplary immunoassays include fluorescent immunosorbent assay (FIA), a chemiluminescent immunoassay (CIA), a radioimmunoassay (MA), multiplex immunoassay, a protein/peptide array immunoassay, a solid phase radioimmunoassay (SPRIA), an indirect immunofluorescence assay (IIF), an enzyme linked immunosorbent assay (ELISA) and a particle based multianalyte test (PMAT) or a Dot Blot assay.

In some embodiments, the ELISA can be a sandwich ELISA. In some embodiments, the sandwich ELISA can include the initial step of immobilizing an isolated KLHL12 fragment of this disclosure on a solid support as disclosed herein. For example, an isolated KLHL12 fragment can be immobilized on a wall of a microtiter plate well or of a cuvette. In some embodiments, contacting the sample from the subject with an isolated KLHL12 fragment can include exposing the sample to the isolated KLHL12 fragment.

In some embodiments, the ELISA can be a direct ELISA. In some embodiments, the direct ELISA can include the initial step of immobilizing an isolated KLHL12 fragment on any of the solid supports disclosed herein. For example, an isolated KLHL12 fragment can be immobilized to a wall of a microtiter plate well or of a cuvette. In some embodiments, contacting the sample from the subject with an isolated KLHL12 fragment can include exposing the anti-KLHL12 antibody contained in the patient's sample to the immobilized isolated KLHL12 fragment. Any of the immunoassays disclosed herein (see above) and in the art can be used or modified to be used, in any of the methods disclosed herein.

As used herein, the term “particle based multianalyte test (PMAT)” is intended to mean an immunoassay that allows simultaneous measurement of two or more analytes in a single assay. For example, in PMAT, different types of particles are used simultaneously, with each type having immobilized a specific binding partner for a specific molecule species on the surface of its particles. In a solution, the analyte molecules to be detected are bound to their binding partners on the corresponding particle type. The bonds are then detected optically through the addition of a secondary marker that marks all particle-bound analyte molecules of the multiplex assay. A PMAT can be performed using a variety of formats known in the art, such as flow cytometry, a capture sandwich immunoassay or a competitive immunoassay. For example, using a dual-laser flow-based detection instrument, the binding of analyte fractions, such as autoantibodies, can be detected through the fluorescence of the secondary marker. In some embodiments, the PMAT particle can be a bead. In effecting the PMAT, the presence of one or more autoantibodies specifically associated with an autoimmune disease can be identified and the patient can be diagnosed with the autoimmune disease that is specifically associated with the autoantibody identified by the PMAT.

In some embodiments, a Dot-Blot or line immunoassay (LIA) can be used to detect anti-KLHL12 antibody in a biological sample. Methods and protocols for dot blot are well known in the art, including estimating polypeptide concentration. See Joint ProteomicS Laboratory (JPSL) of the Ludwig Institute for Cancer Research, Estimating protein concentration by dot blotting of multiple samples, Cold Spring Harbor Protocols, New York (2006).

The immunoassay can further include blocking steps, washing steps and additionally or alternatively, elution steps. Blocking steps can include contacting a solid support of the immunoassay in a blocking buffer for a sufficient time and temperature to allow blocking. Exemplary blocking buffers are identified above. Washing steps include contacting a solid support of the immunoassay with a wash buffer to remove non-specific binding of polypeptides to the solid support. Exemplary washing buffers are described above. Elution buffers can include any of a variety of elution buffers known in the art or disclosed herein. Elution buffers include, for example, a 0.1 M glycine:HCl solution between pH 2.5 and 3. Polypeptide complexes can be eluted from the solid support of the immunoassay to aid in detection and measurement of, for example, KLHL12:anti-KLHL12 antibody complexes.

The detection is performed on a solid support. Exemplary solid support includes a bead, sphere, particle, membrane, chip, slide, plate, well and test tube as disclosed herein. In some embodiments, the bead, sphere or particle comprises micrometer or nanometer dimensions. In some embodiments, the membrane is selected from the group consisting of nitrocellulose, nylon, polyvinylidene fluoride (PVDF) and polyvinylidene difluoride.

### SEQUENCES

The sequences in Table 2 are illustrative amino acid sequences of the isolated KLHL12 fragments described herein.

TABLE 2

| SEQ ID NO | Sequence |
|---|---|
| 1 | CYDPIIDSWEVVTSM |
| 2 | IECYDPIIDSWEVVT |
| 3 | YDPIIDSWEVVTSMG |
| 4 | SIECYDPIIDSWEVV |
| 5 | ECYDPIIDSWEVVTS |
| 6 | VVASGVIYCLGGYDG |
| 7 | GHWTNVTPMATKRSG |
| 8 | AGVALLNDHIYVVGG |
| 9 | ASGVIYCLGGYDGLN |
| 10 | GAGVALLNDHIYVVG |
| 11 | MGGIMAPKDIMTNTH |
| 12 | GLVVASGVIYCLGGY |
| 13 | SGVIYCLGGYDGLNI |
| 14 | LQYVRMPLLTPRYIT |
| 15 | MTTPRCYVGATVLRG |
| 16 | GLAGATTLGDMIYVS |
| 17 | RIYVIGGYDGRSRLS |
| 18 | ECLDYTADEDGVWYS |
| 19 | GVWYSVAPMNVRRGL |
| 20 | DSWTTVTSMTTPRCY |
| 21 | YNIRTDSWTTVTSMT |
| 22 | GGFDGSRRHTSMERY |
| 23 | RSGAGVALLNDHIYV |
| 24 | LNDHIYVVGGFDGTA |
| 25 | IECYDPIIDSWEVVTSMG |
| 26 | GSGSGSG |
| 27 | YPYDVPDYAG |

### Example I

### Selecting KLHL12 Fragment Candidates Via Epitope Mapping of KLHL12 Protein

This example illustrates the process of selecting KLHL12 fragment candidates via epitope mapping of KLHL12 protein.

Two pools of sera were utilized for epitope mapping. The first pool consisted of sera containing anti-KLHL12 antibodies and the second pool consisted of sera from systemic lupus erythematous (SLE) patients negative for anti-KLHL12 antibodies. The two pools were used to perform epitope mapping on custom sequential overlapping KLHL12 fragments synthesized on a solid phase matrix (PEPperPRINT GmbH, Heidelberg, Germany).

The sequence of KLHL12 protein (NCBI Gene ID: 59349) was elongated with neutral GSGSGSG (SEQ ID NO:26) linkers at the C- and N-terminus to avoid truncated KLHL12 fragments. The elongated KLHL12 protein sequence was translated into 15 amino acid KLHL12 fragments with a peptide-peptide overlap of 14 amino acids. The resulting KLHL12 fragments were printed in duplicate and framed by additional control peptides (YPYDVPDYAG (SEQ ID NO:27)). One copy of the microarrays, including both the resulting KLHL12 fragments and the control peptides, was then incubated with the pool of sera containing anti-KLHL12 antibodies. The other copy of the microarrays was incubated with the pool of sera negative for anti-KLHL12 antibodies.

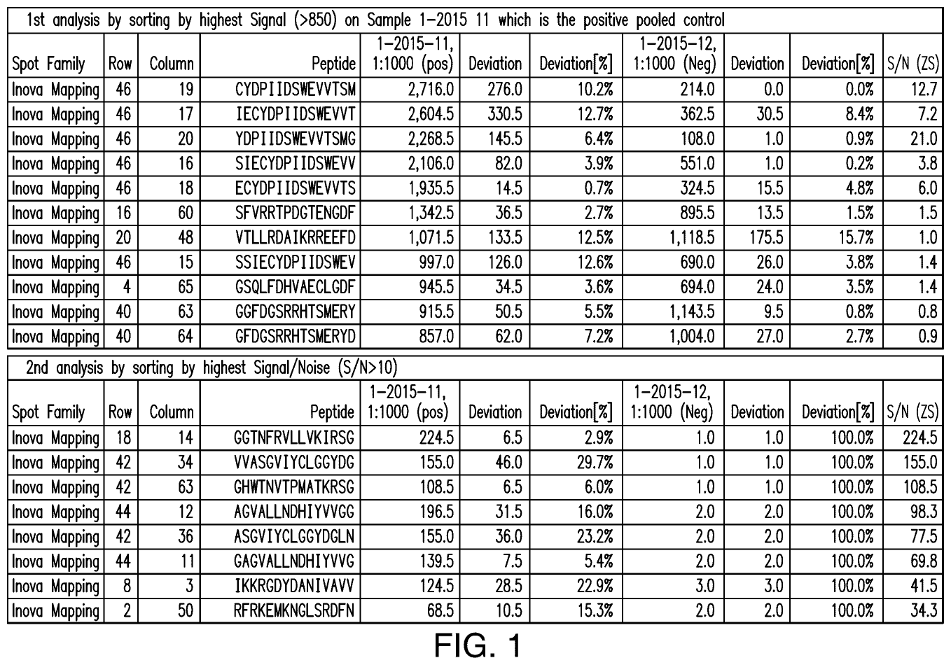

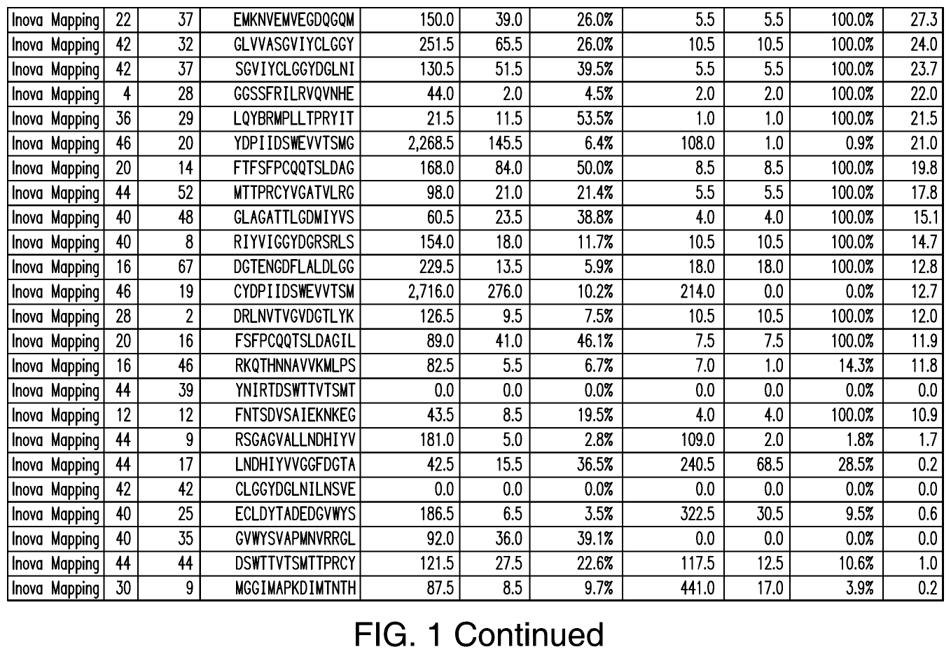

Signal intensity of each spot in the two microarrays was quantified by an imaging instrumentation. Spots with high signal intensity or high signal-to-noise ratio shown in FIG. 1 suggested KLHL12 fragment candidates for further analysis. Those KLHL12 fragment candidates were synthesized and evaluated via several solid-phase assays, including ELISA and bead-based technologies.

This example demonstrates that the KLHL12 fragments disclosed herein, which have the capacity to specifically bind anti-KLHL12 antibodies, are systematically screened and evaluated.

### Example II

### Performance of Isolated KLHL12 Fragments in Detecting Anti-KLHL12 Antibodies in Sera Specimens from PBC Patients

This example illustrates the capacity of isolated KLHL12 fragments to detect anti-KLHL12 antibodies in sera specimens from PBC patients.

The KLHL12 fragments consisting of the amino acid sequence of SEQ ID NO:1 were labeled with biotin either at the C-terminus (for P2623-1) or at the N-terminus (for P2738-1). The labeled KLHL12 fragments were further coated at 5 ug/ml to either Costar high-binding microwell plate (for P2623-1) or Inova streptavidin coated plate (for P2738-1) using standard Inova proprietary protocols and materials.

Detection of anti-KLHL12 antibodies in sera specimens was performed by (1) making a 1:101 dilution of the sera specimens in horseradish peroxidase (HR) sample diluent, (2) applying 100 ul of the diluted specimen to each well, and (3) incubating the diluted specimen with the well containing immobilized KLHL12 fragments for 30 minutes at room temperature. At the completion of the incubation, the wells were washed 3 times with 200-300 ul HRP wash solution to wash away unbound anti-KLHL12 antibodies. Each well was then incubated with 100 ul of HRP-conjugated goat-anti-human IgG for 30 minutes at room temperature. At the completion of the incubation, the wells were washed 3 times with 200-300 ul HRP wash solution to wash away unbound HRP-conjugated goat-anti-human IgG. Each well was then incubated with 100 ul of TMB (3,3′, 5,5;-tetramethylbenzidine) chromagen for 30 minutes. The reaction was stopped by the addition of 100 ul of 0.1N H₂SO₄ stop solution. The intensity of the developed color was measured spectrophotometrically at 450/620 nm to assess reactivity.

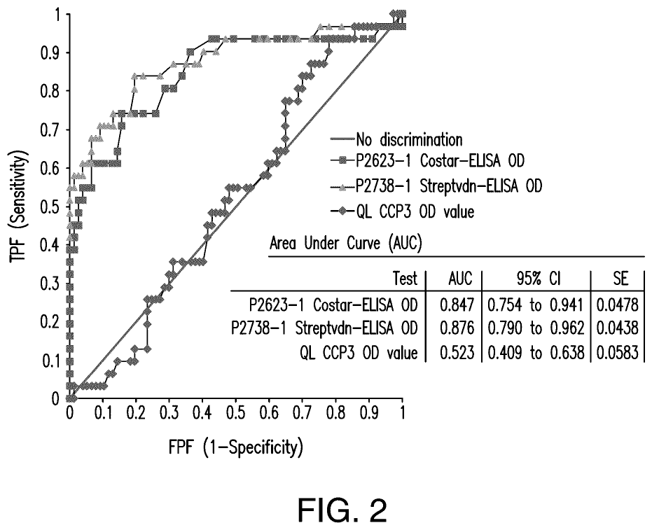

To demonstrate the preferential reactivity of the sera specimens from PBC patients to KLHL12 fragments, the sera specimens were also tested for reactivity to Cyclic Citrullinated Peptide (CCP), a target for antibodies present in many patients with rheumatoid arthritis (RA). The CCP coated plate used herein was obtained from QUANTA Lite® CCP3 IgG ELISA kits (Inova Diagnostics, San Diego, CA). Receiver operating characteristics (ROC) analysis of the results clearly showed the preferential binding of the sera specimens from PBC patients to the KLHL12 fragments. In contrast, the sera specimens from PBC patients showed only negligible binding to the RA associated antigen, CCP. See FIG. 2.

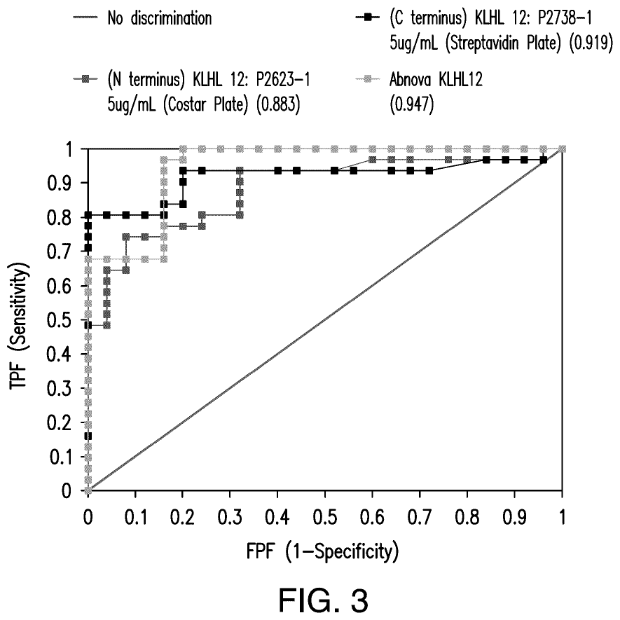

To demonstrate the effectiveness of KLHL12 fragments at detecting anti-KLHL12 antibodies in sera specimens from PBC patients, the sera specimens were also tested for reactivity to a full-length recombinant KLHL12 protein with GST-tag at the N-terminus. The KLHL12 protein was commercially obtained from Abnova Corporation, Taipei, Taiwan. Receiver operating characteristics (ROC) analysis of the results clearly showed the KLHL12 fragments were as effective as the full-length KLHL12 proteins at binding anti-KLHL12 antibodies in the sera specimens from PBC patients. See FIG. 3.

Sera specimens from 254 PBC patients from the Liver Unit of the Hospital Clinic of Barcelona, Spain and 40 PBC patients from Saint-Antoine Hospital of Paris, France were collected. The collected specimens were tested for the prevalence of anti-KLHL12 antibodies using QUANTA Lite® ELISA kits (Inova Diagnostics, San Diego, CA). The amino acid sequence of the KLHL12 fragment used was IECYDPIIDSWEVVTSMG (SEQ ID NO:25), which consists of the amino acid sequence of SEQ ID NO:2 with three additional amino acids at the C-terminus. The KLHL12 fragment was labeled with biotin at the C-terminus.

Anti-KLHL12 antibodies were positive in 22.8% of the Spain cohort of PBC patients and 20% of the French cohort of PBC patients, demonstrating that KLHL12 fragments detect anti-KLHL12 antibodies in sera specimens from PBC patients from different geographic locales.

This example demonstrates that the KLHL12 fragments disclosed herein can diagnose PBC via detecting anti-KLHL12 antibodies in sera specimens from PBC patients.

### Example III

### Using an Isolated KLHL12 Fragment to Increase the Sensitivity of Diagnosing PBC

This example illustrates that the addition of anti-KLHL12 antibody testing using an KLHL12 fragment disclosed herein can increase the sensitivity of diagnosing PBC. The amino acid sequence of the KLHL12 fragment used in this example was IECYDPIIDSWEVVTSMG (SEQ ID NO:25), which consists of the amino acid sequence of SEQ ID NO:2 with three additional amino acids at the C-terminus. The KLHL12 fragment was labeled with biotin at the C-terminus.

Sera specimens from a total of 487 patients with clinically documented PBC or PBC/AIH overlap diagnosed according to European Association for the Study of the Liver (EASL) guidelines were collected at five expert clinical sites (Barcelona, Spain; Salamanca, Spain; Calgary, Canada; Edmonton, Canada; Warsaw, Poland). The collected specimens were tested for the presence of antibodies to Hexokinase 1(HK-1) and KLHL12 using QUANTA Lite® ELISA kits (Inova Diagnostics, San Diego, CA). All sera specimens were also tested for anti-MIT3 autoantibodies.

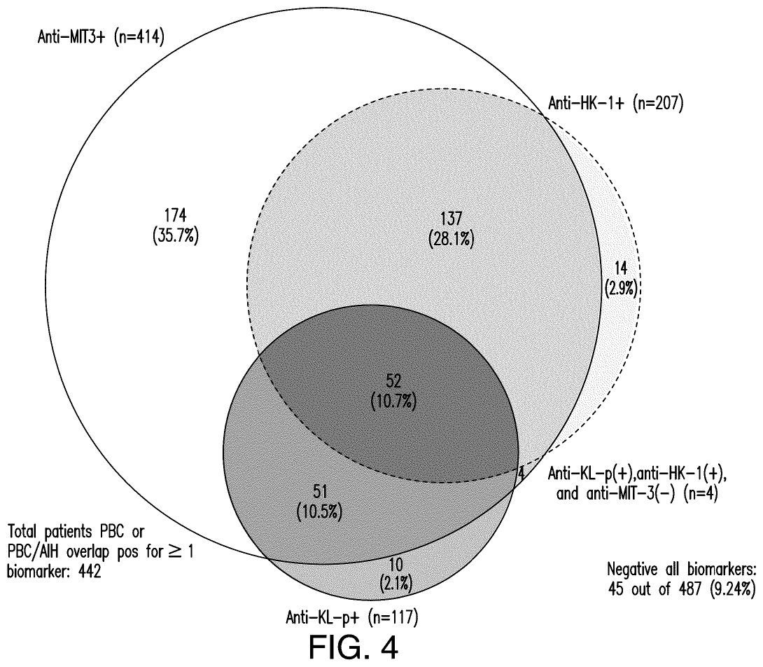

The addition of anti-HK-1 and anti-KLHL12 antibody testing to anti-MIT3 antibody testing increased the sensitivity of detecting PBC from 85.0% for anti-MIT3 alone to 90.8% for the combination of anti-MIT3, anti-HK-1 and anti-KLHL12 for the sample cohort. See FIG. 4.

This example demonstrates that the KLHL12 fragments disclosed herein can be used to increase the sensitivity of diagnosing PBC.

### Example IV

### Using an Isolated KLHL12 Fragment to Increase the Clinical Confidence of Diagnosing PBC

This example illustrates that the addition of anti-KLHL12 antibody testing using an KLHL12 fragment disclosed herein can increase the clinical confidence of diagnosing PBC. The amino acid sequence of the KLHL12 fragment used in this example was IECYDPIIDSWEVVTSMG (SEQ ID NO:25), which consists of the amino acid sequence of SEQ ID NO:2 with three additional amino acids at the C-terminus. The KLHL12 fragment was labeled with biotin at the C-terminus.

Sera specimens tested here were from the 40 PBC patients from Saint-Antoine Hospital of Paris, France as described in Example II above. The specimens were tested for the presence of antibodies to MIT3, LKM-1, SLA, LC-1, sp100, gp210, HK-1, and KLHL12 using Aptiva® Liver Reagent (Research Use Only, Inova Diagnostics, San Diego, CA).

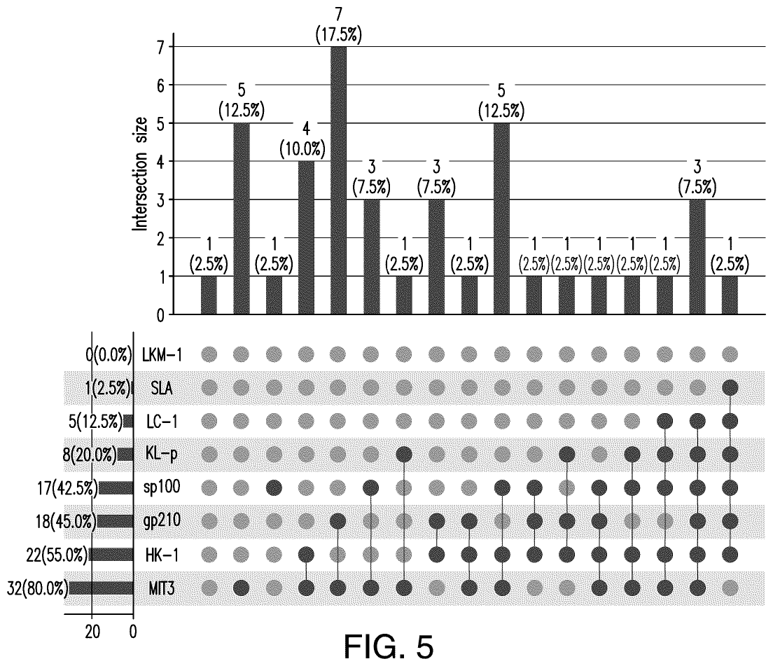

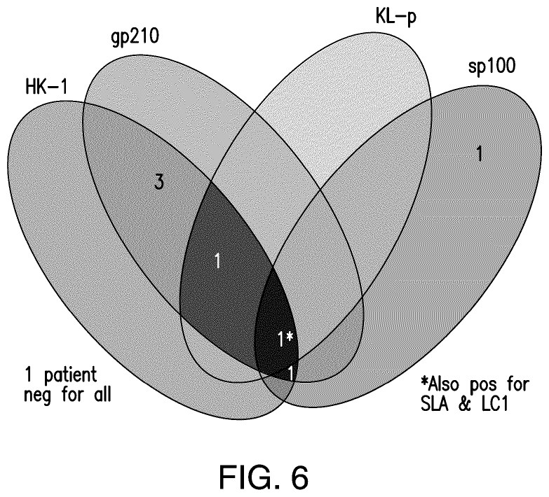

The frequency and overlap of autoimmune liver disease-associated biomarkers detected in the 40 PBC patients are shown in FIG. 5. Anti-KLHL12 antibodies were shown to be present along with other PBC-specific biomarkers. Moreover, among MIT3-negative PBC patients, 25% ( 2/8) were positive for anti-KLHL12 antibodies, as well as anti-gp210 and anti-HK-1 antibodies. See FIG. 6.

This example demonstrates that the KLHL12 fragments disclosed herein can be included as a component of a multi-analyte assay. Such inclusion increases the clinical confidence of diagnosing PBC.

1-12. (canceled)

13. A kit comprising:
(a) one or more isolated polypeptides comprising a Kelch-like 12 (KLHL12) fragment having an amino acid sequence selected from the group consisting of SEQ ID NOS:1-25, wherein the amino acid sequence begins at the first recited amino acid residue; and
(b) a detection probe specific to an anti-KLHL12 antibody.

14. The kit of claim 13, wherein the KLHL12 fragment comprises 15-622 amino acid residues.

15. The kit of claim 13, wherein the KLHL12 fragment comprises 15, 20, 25, 30, 35, 40, 45 or 50 amino acid residues.

16. (canceled)

17. The kit of claim 13, wherein the KLHL12 fragment specifically binds to an anti-KLHL12 antibody and the binding exhibits a standard score for a signal-to-noise ratio of 25 or less (ZS).

18. The kit of claim 17, wherein the anti-KLHL12 antibody comprises an autoantibody.

19-20. (canceled)

21. The kit of claim 13, wherein the detection probe comprises an antibody, an antibody specific binding polypeptide or a functional fragment of an antibody or an antibody specific binding polypeptide.

22. The kit of claim 21, wherein the antibody or function fragment thereof comprises anti-IgG.

23. The kit of claim 21, wherein the antibody specific binding polypeptide or functional fragment thereof comprises protein A or protein G.

24. The kit of claim 13, wherein the detection probe comprises a reporter tag or label selected from the group consisting of a fluorophore, enzyme, chemiluminescent moiety, radioactive moiety, organic dye, and small molecule.

25-26. (canceled)

27. The kit of claim 24, wherein the label is a fluorescent label.

28. The kit of claim 27, wherein the fluorescent label is phycoerytherin (PE), horseradish peroxidase or alkaline phosphatase.

29. The kit of claim 24, wherein the reporter tag comprises a ligand or particle.

30-31. (canceled)

32. The kit of claim 13, further comprising a solid support, control or ancillary reagent.

33. The kit of claim 32, wherein the solid support is selected from the group consisting of a bead, sphere, particle, membrane, chip, slide, plate, well and test tube.

34-35. (canceled)

36. The kit of claim 13, wherein the one or more isolated polypeptides is conjugated to the solid support.

37. The kit of claim 32, wherein the control comprises an antibody or functional fragment thereof specific to an isolated polypeptide having an amino acid sequence selected from the group consisting of SEQ ID NOS:1-25.

38. (canceled)

39. The kit of claim 32, wherein the ancillary reagent is selected from the group consisting of incubation buffer, wash buffer, detection buffer and detection instrument.

40. A method comprising:
(a) contacting a biological sample with one or more isolated polypeptides comprising a Kelch-like 12 (KLHL12) fragment having amino acid sequence selected from the group consisting of SEQ ID NOS:1-25, and
(b) detecting the presence of an anti-KLHL12 antibody in the biological sample using a detection probe specific to an anti-KLHL12 antibody, wherein the presence of the bound anti-KLHL12 antibody is indicative of primary biliary cirrhosis (PBC).

41-100. (canceled)

101. The method of claim 40, wherein the sample is from a subject suspected of having PBC or is from a subject seronegative for known PBC autoantibodies.

102. An isolated polypeptide comprising a Kelch-like 12 (KLHL12) fragment comprising an amino acid sequence selected from the group consisting of SEQ ID NOS:1-25, wherein the amino acid sequence begins at the first recited amino acid residue.


SEQUENCE LISTING (27 sequences)

SEQ ID NO: 1 - protein, 15 aa, Artificial Sequence
  KLHL12 fragment
CYDPIIDSWEVVTSM

SEQ ID NO: 2 - protein, 15 aa, Artificial Sequence
  KLHL12 fragment
IECYDPIIDSWEVVT

SEQ ID NO: 3 - protein, 15 aa, Artificial Sequence
  KLHL12 fragment
YDPIIDSWEVVTSMG

SEQ ID NO: 4 - protein, 15 aa, Artificial Sequence
  KLHL12 fragment
SIECYDPIIDSWEVV

SEQ ID NO: 5 - protein, 15 aa, Artificial Sequence
  KLHL12 fragment
ECYDPIIDSWEVVTS

SEQ ID NO: 6 - protein, 15 aa, Artificial Sequence
  KLHL12 fragment
VVASGVIYCLGGYDG

SEQ ID NO: 7 - protein, 15 aa, Artificial Sequence
  KLHL12 fragment
GHWTNVTPMATKRSG

SEQ ID NO: 8 - protein, 15 aa, Artificial Sequence
  KLHL12 fragment
AGVALLNDHIYVVGG

SEQ ID NO: 9 - protein, 15 aa, Artificial Sequence
  KLHL12 fragment
ASGVIYCLGGYDGLN

SEQ ID NO: 10 - protein, 15 aa, Artificial Sequence
  KLHL12 fragment
GAGVALLNDHIYVVG

SEQ ID NO: 11 - protein, 15 aa, Artificial Sequence
  KLHL12 fragment
MGGIMAPKDIMTNTH

SEQ ID NO: 12 - protein, 15 aa, Artificial Sequence
  KLHL12 fragment
GLVVASGVIYCLGGY

SEQ ID NO: 13 - protein, 15 aa, Artificial Sequence
  KLHL12 fragment
SGVIYCLGGYDGLNI

SEQ ID NO: 14 - protein, 15 aa, Artificial Sequence
  KLHL12 fragment
LQYVRMPLLTPRYIT

SEQ ID NO: 15 - protein, 15 aa, Artificial Sequence
  KLHL12 fragment
MTTPRCYVGATVLRG

SEQ ID NO: 16 - protein, 15 aa, Artificial Sequence
  KLHL12 fragment
GLAGATTLGDMIYVS

SEQ ID NO: 17 - protein, 15 aa, Artificial Sequence
  KLHL12 fragment
RIYVIGGYDGRSRLS

SEQ ID NO: 18 - protein, 15 aa, Artificial Sequence
  KLHL12 fragment
ECLDYTADEDGVWYS

SEQ ID NO: 19 - protein, 15 aa, Artificial Sequence
  KLHL12 fragment
GVWYSVAPMNVRRGL

SEQ ID NO: 20 - protein, 15 aa, Artificial Sequence
  KLHL12 fragment
DSWTTVTSMTTPRCY

SEQ ID NO: 21 - protein, 15 aa, Artificial Sequence
  KLHL12 fragment
YNIRTDSWTTVTSMT

SEQ ID NO: 22 - protein, 15 aa, Artificial Sequence
  KLHL12 fragment
GGFDGSRRHTSMERY

SEQ ID NO: 23 - protein, 15 aa, Artificial Sequence
  KLHL12 fragment
RSGAGVALLNDHIYV

SEQ ID NO: 24 - protein, 15 aa, Artificial Sequence
  KLHL12 fragment
LNDHIYVVGGFDGTA

SEQ ID NO: 25 - protein, 18 aa, Artificial Sequence
  KLHL12 fragment
IECYDPIIDSWEVVTSMG

SEQ ID NO: 26 - protein, 7 aa, Artificial Sequence
  Linker
GSGSGSG

SEQ ID NO: 27 - protein, 10 aa, Artificial Sequence
  Control peptide
YPYDVPDYAG